(19) United States
(12) Patent Application Publication
(10) Pub. No.: US 2024/0108697 A1
(43) Pub. Date: Apr. 4, 2024

(54) ANTIMICROBIAL COMPOSITIONS

(71) Applicant: Matoke Holdings Limited, Southmoor Abingdon Oxfordshire (GB)

(72) Inventors: Stewart GREEN, Southmoor Abingdon Oxfordshire (GB); Mark BROUGHTON, Southmoor Abingdon Oxfordshire (GB); Wendy RICHINGS-BARROW, Southmoor Abingdon Oxfordshire (GB); Ian HARDING, Southmoor Abingdon Oxfordshire (GB)

(21) Appl. No.: 18/282,403

(22) PCT Filed: Mar. 16, 2021
(86) PCT No.: PCT/GB2021/050655

§ 371 Date: Sep. 15, 2023

(30) Foreign Application Priority Data
Mar. 16, 2020 (GB) 2003774.3
May 4, 2020 (GB) 2006545.4

(51) Int. Cl. A61K 38/44; A61K 9/06; A61K 47/10; A61K 47/22; A61K 47/26; A61P 17/02; A61P 31/14

(52) U.S. Cl. CPC A61K 38/443; A61K 9/06; A61K 47/10; A61K 47/22; A61K 47/26; A61P 17/02; A61P 31/14; C12Y 101/03004

(57) ABSTRACT

Liquid or gel antimicrobial compositions are described. The compositions comprise enzyme that is able to convert a substrate to release hydrogen peroxide; and substrate for the enzyme. The compositions do not comprise sufficient free water to allow the enzyme to convert the substrate, or have a water activity (aw) of 0.7 or less. The compositions may comprise polymer, and there may be greater than 50%, by weight, of the polymer and/or less than 20%, by weight, of sugar in the composition. The compositions may comprise non-aqueous solvent, no honey and 5% or less, by weight, of the substrate in the composition. There may be greater than 50%, by weight, of the non-aqueous solvent and/or there is less than 20%, by weight, of sugar in the composition.

This invention relates to compositions for generating hydrogen peroxide, and their use in treating infections and wounds.

Honey has been used for treatment of microbial infections since ancient times. In recent years there has been a resurgence of interest in the therapeutic efficacy of honey, particularly in the area of wound healing. Clinical trials have shown that honey is an effective broad-spectrum antimicrobial agent which is effective against common wound-infecting organisms, such as Pseudomonas aeruginosa, Staphylococcus aureus, Candida albicans and Escherichia coli, and is effective against antibiotic-resistant strains of bacteria. As a natural product, honey also offers an attractive alternative to drug-based treatments.

Many different types of honey have antimicrobial activity. This activity is attributed largely to osmolarity, pH, hydrogen peroxide production and the presence of phytochemical components.

The applicant has appreciated that the antimicrobial effects of honey can be greatly enhanced and controlled by adding glucose oxidase to honey, and that compositions comprising honey and added glucose oxidase are applicable in the treatment of a number of infections, and notably in the treatment of infections caused by biofilms (see WO 2015/166197, WO 2016/083798 and WO 2016/124926).

However, because honey is a natural product, its composition can vary greatly depending on its source. For example, the difference in antimicrobial potency among honeys can be more than one hundred-fold, depending on the geographical, seasonal and botanical source of the honey, as well as the harvesting, processing and storage conditions. Consequently, the antimicrobial efficacy may also vary depending on the type of honey used. Furthermore, honey may also contain other components, such as allergens e.g. trace amounts of pollen, which may cause adverse reactions when applied to certain subjects and make it unsuitable for certain pharmaceutical applications. There is also considerable variability in physical characteristics such as viscosity and colour.

Honey is sticky and can be difficult to apply and remove from a patient. Honey may also require processing such that it is in a suitable form for application to subjects, which can add cost and complexity to the production process. Such processing may include creaming or pasteurisation.

Consequently, there is a desire to provide improved compositions which provide enhanced antimicrobial efficacy compared to honey, and which also overcome some of honey's disadvantages. There is also a desire to provide compositions with improved stability and which have the ability to provide antimicrobial activity over an extended period of time.

In a broad sense, the invention provides a composition comprising an enzyme that is able to convert a substrate to release hydrogen peroxide and a substrate for the enzyme.

The invention also provides a method of making a composition comprising combining an enzyme that is able to convert a substrate to release hydrogen peroxide and a substrate for the enzyme.

References herein to “enzyme” encompass one or more enzymes. For example, in some embodiments, compositions of the invention may comprise a plurality of enzymes that are able to convert a substrate to release hydrogen peroxide. In some embodiments, compositions of the invention may comprise only one enzyme that is able to convert a substrate to release hydrogen peroxide.

Preferably, the enzyme is a purified enzyme. The term “purified enzyme” is used herein to include an enzyme preparation in which the enzyme has been separated from at least some of the impurities originally present when the enzyme was produced. Preferably impurities that have been removed or reduced include those that would otherwise interfere with the ability of the enzyme to convert the substrate to release hydrogen peroxide.

It may not always be necessary or desirable that the purified enzyme is at a high level of purity provided that the enzyme is able to convert the substrate to release hydrogen peroxide. In some circumstances, it may be desirable to use a relatively crude enzyme preparation. Examples of suitable purity levels include at least 10%, 20%, 30%, 40%, 50%, 60%, 70%, 80%, or 90% pure (mass purity). Preferably, the enzyme is at least 95% pure. Even more preferably, the enzyme is at least 98% pure. Most preferably, the enzyme is at least 99% pure.

The enzyme may have been produced by recombinant or non-recombinant means, and may be a recombinant or non-recombinant enzyme. The enzyme may be purified from a microbial source, preferably from a non-genetically modified microbe.

The level of purity of the enzyme may be selected as appropriate depending on the intended use of the composition. For medical use, a medical grade or medical device grade of purity should be used. For pharmaceutical use, a pharmaceutical grade of purity should be used.

Compositions of the invention may comprise sufficient enzyme and substrate to provide for release of hydrogen peroxide at a specific level or concentration.

Compositions of the invention may provide for release of hydrogen peroxide at a concentration of at least 150 μM, optionally at one hour, 24 hours, or 72 hours following a 1:1 (by weight) dilution of the composition with water.

Compositions of the invention may provide for release of hydrogen peroxide at a concentration of at least 250 μM, optionally at one hour, 24 hours, or 72 hours following a 1:1 (by weight) dilution of the composition with water.

Compositions of the invention may provide for release of hydrogen peroxide at a concentration of at least 500 μM, optionally at one hour, or 24 hours, or 72 hours following a 1:1 (by weight) dilution of the composition with water.

Compositions of the invention may provide for release of hydrogen peroxide at a concentration of at least 750 μM, optionally at one hour, 24 hours or 72 hours, following a 1:1 (by weight) dilution of the composition with water.

Compositions of the invention may provide for release of hydrogen peroxide at a concentration of at least 1,000 μM, optionally at one hour, 24 hours, or 72 hours following a 1:1 (by weight) dilution of the composition with water.

Compositions of the invention may provide for release of hydrogen peroxide at a concentration of at least 1,500 μM, optionally at one hour, 24 hours, or 72 hours following a 1:1 (by weight) dilution of the composition with water.

Compositions of the invention may provide for release of hydrogen peroxide at a concentration of at least 2,000 μM, optionally at one hour, 24 hours, or 72 hours following a 1:1 (by weight) dilution of the composition with water.

Compositions of the invention may provide for release of hydrogen peroxide at a concentration of at least 5,000 μM, optionally at one hour, 24 hours, or 72 hours following a 1:1 (by weight) dilution of the composition with water.

Compositions of the invention may provide for release of hydrogen peroxide at a concentration of at least 10,000 μM, optionally at one hour, 24 hours, or 72 hours following a 1:1 (by weight) dilution of the composition with water.

Compositions of the invention may provide for release of hydrogen peroxide at a concentration of at least 7,500 μM, optionally at one hour, 24 hours, or 72 hours following a 1:1 (by weight) dilution of the composition with water.

Compositions of the invention may provide for release of hydrogen peroxide at a concentration of 5,000 μM or less, optionally at one hour, 24 hours, or 72 hours following a 1:1 (by weight) dilution of the composition with water.

Compositions of the invention may provide for release of hydrogen peroxide at a concentration of 2,500 μM or less, optionally at one hour, 24 hours, or 72 hours following a 1:1 (by weight) dilution of the composition with water.

Compositions of the invention may provide for release of hydrogen peroxide at a concentration of 2,000 μM or less, optionally at one hour, 24 hours, or 72 hours following a 1:1 (by weight) dilution of the composition with water.

Compositions of the invention may provide for release of hydrogen peroxide at a concentration of 1,000 μM or less, optionally at one hour, 24 hours, or 72 hours following a 1:1 (by weight) dilution of the composition with water.

Compositions of the invention may provide for release of hydrogen peroxide at a concentration of 500 μM or less, optionally at one hour, 24 hours, or 72 hours following a 1:1 (by weight) dilution of the composition with water.

Compositions of the invention may provide for release of hydrogen peroxide at a concentration of 10,000 μM or less, optionally at one hour, 24 hours, or 72 hours following a 1:1 (by weight) dilution of the composition with water.

Compositions of the invention may provide for release of hydrogen peroxide at a concentration of 20,000 μM or less, optionally at one hour, 24 hours, or 72 hours following a 1:1 (by weight) dilution of the composition with water.

Compositions of the invention may provide for release of hydrogen peroxide at a concentration of 30,000 μM or less, optionally at one hour, 24 hours, or 72 hours following a 1:1 (by weight) dilution of the composition with water.

Compositions of the invention may provide for release of hydrogen peroxide at a concentration of 50,000 μM or less, optionally at one hour, 24 hours, or 72 hours following a 1:1 (by weight) dilution of the composition with water.

Compositions of the invention may provide for release of hydrogen peroxide at a concentration of 80,000 μM or less, optionally at one hour, 24 hours, or 72 hours following a 1:1 (by weight) dilution of the composition with water.

Compositions of the invention may provide for release of hydrogen peroxide at a concentration of 100,000 μM or less, optionally at one hour, 24 hours, or 72 hours following a 1:1 (by weight) dilution of the composition with water.

Compositions of the invention may provide for release of hydrogen peroxide at a concentration of 500 to 100,000 μM, optionally at one hour, 24 hours, or 72 hours following a 1:1 (by weight) dilution of the composition with water.

Compositions of the invention may provide for release of hydrogen peroxide at a concentration of 500 to 50,000 μM, optionally at one hour, 24 hours, or 72 hours following a 1:1 (by weight) dilution of the composition with water.

Compositions of the invention may provide for release of hydrogen peroxide at a concentration of 500 to 10,000 μM, optionally at one hour, 24 hours, or 72 hours following a 1:1 (by weight) dilution of the composition with water.

Compositions of the invention may provide for release of hydrogen peroxide at a concentration of 500 to 7,500 μM, optionally at one hour, 24 hours, or 72 hours following a 1:1 (by weight) dilution of the composition with water.

Compositions of the invention may provide for release of hydrogen peroxide at a concentration of 500 to 5,000 μM, optionally at one hour, 24 hours, or 72 hours following a 1:1 (by weight) dilution of the composition with water.

Compositions of the invention may provide for release of hydrogen peroxide at a concentration of 500 to 2,500 μM, optionally at one hour, 24 hours, or 72 hours following a 1:1 (by weight) dilution of the composition with water.

Compositions of the invention may provide for release of hydrogen peroxide at a concentration of 750 to 2,500 μM, optionally at one hour, 24 hours, or 72 hours following a 1:1 (by weight) dilution of the composition with water.

Compositions of the invention may provide for release of hydrogen peroxide at a concentration of 500 to 2,000 μM, optionally at one hour, 24 hours, or 72 hours following a 1:1 (by weight) dilution of the composition with water.

Compositions of the invention may provide for release of hydrogen peroxide at a concentration of 150 to 2,000 μM, optionally at one hour, 24 hours, or 72 hours following a 1:1 (by weight) dilution of the composition with water.

Compositions of the invention may provide for release of hydrogen peroxide at a concentration of 150 to 1,000 μM, optionally at one hour, 24 hours, or 72 hours following a 1:1 (by weight) dilution of the composition with water.

Compositions of the invention may provide for release of hydrogen peroxide at a concentration of 150 to 500 μM, optionally at one hour, 24 hours, or 72 hours following a 1:1 (by weight) dilution of the composition with water.

The level of hydrogen peroxide produced on dilution of compositions of the invention may be sustained at particular levels for a period of time. For example, the level of hydrogen peroxide may be 200 μM to 5,000 μM, preferably at least 400 μM to 3,000 μM, more preferably at least 750 μM to 2500 μM for at least 72 hours following dilution of the composition e.g. following a 1:1 (by weight) dilution of the composition with water. Appropriate levels may ensure antimicrobial, but non-cytotoxic activity.

In some embodiments, compositions of the invention may have a particular hydrogen peroxide release profile following dilution. For instance, upon dilution, there may be an initial burst of hydrogen peroxide release, followed by a lower release rate sustained for at least 72 hours. In one example, the composition may provide for release of hydrogen peroxide at a concentration of 1500 μM to 2500 μM at one hour following dilution (e.g. a 1:1, by weight dilution of the composition with water), 1000 μM to 2000 μM at 24 hours following dilution and then 200 μM to 1000 μM between 48 hours and 72 hours following dilution. In another example, the composition may provide for release of hydrogen peroxide at a concentration of about 2000 μM at one hour following dilution (e.g. a 1:1, by weight dilution of the composition with water), 1200 μM to 1500 μM at 24 hours following dilution and then 400 μM to 700 μM between 48 hours and 72 hours following dilution.

The levels of hydrogen peroxide described herein, prior to and/or following dilution may be assessed at ambient temperature, such as normal temperature and pressure (NTP; 20° C. and 1 atm).

Levels of hydrogen peroxide may be established using a method as described in Example 1. Alternatively, levels of hydrogen peroxide may be quantified following the method of Kerkvliet 1996 and Serrano et al., 2004, using Merckoquant test strip (no. 10011; Merck, Germany).

In some embodiments, the composition may not comprise sufficient free water to allow the enzyme to convert the substrate. If the composition does not comprise sufficient free water to allow the enzyme to convert the substrate, hydrogen peroxide production may only occur once it has been diluted by water and there is sufficient free water to allow the enzyme to convert the substrate. Addition of water may thus initiate hydrogen peroxide production.

The skilled person would understand a composition that does not comprise sufficient free water to allow the enzyme to convert the substrate, encompasses a composition that contains a trace amount (or low levels) of free water that may allow a trace amount (or low levels) of hydrogen peroxide to be produced.

Before dilution, hydrogen peroxide may be present at a concentration of less than 10 ppm, 6 ppm or less, 5 ppm or less, 3 ppm or less, or 2 ppm or less. Hydrogen peroxide may be present in the composition at a concentration of 120 μM or less, preferably 100 μM or less, more preferably 80 μM or less. Low levels of hydrogen peroxide before dilution may advantageously improve the shelf life of compositions of the invention. Higher levels of hydrogen peroxide may result in loss of enzyme activity over time. This may be caused by oxidative damage to the enzyme by the hydrogen peroxide being produced.

Once the composition is diluted, hydrogen peroxide may be generated at substantial concentrations. At 1 hour, following a 1:1 dilution (by weight) with water, the level of hydrogen peroxide production may increase by a factor of at least 5, at least 10, at least 20, at least 50, at least 100, at least 200 or at least 300. At 24 hours, following a 1:1 dilution (by weight) with water, the level of hydrogen peroxide production may increase by a factor of at least 5, at least 10, at least 20, at least 50, at least 100, at least 200, or at least 300.

In compositions of the invention, the water activity (a w) may be 0.8 or less, 0.7 or less, 0.6 or less, 0.5 or less, 0.4 or less, or 0.3 or less. For example, the water activity may be 0.2 to 0.8, for example 0.3 to 0.7 or 0.4 to 0.6. A low water activity may be advantageous in preventing microbial proliferation, and it may be advantageous in minimising hydrogen peroxide production prior to activation by dilution.

Water activity is typically measured using a hygrometer, such as a resistive electrolytic hygrometer, a capacitance hygrometer or a dew point hygrometer. Measurement of water activity would typically take place at ambient temperature, such as normal temperature and pressure . Measurement of water activity may take place according to ISO 18787:2017.

Suitable amounts of water will vary depending on the precise components of the composition. Typically, there would be 25% (by weight) or less, preferably 20% or less (by weight) water in the composition, for example, 5%-20%, water. In one embodiment, there is 5 to 17% (by weight) of water. In one embodiment, there is 5 to 15% (by weight) of water. In a preferred embodiment, the amount of water in the composition is 10% or less, preferably 7.5% or less, more preferably 5% or less (by weight). In one embodiment, the amount of water may be 1% to 10% (by weight), such as 2.5% to 7.5% (by weight). In some embodiments, the composition may comprise substantially no water, or only trace amounts of water.

Compositions of the invention are preferably fluid at normal temperature and pressure (20° C. and 1 atm). For example, compositions of the invention are preferably a liquid at normal temperature and pressure. For example, a composition of the invention may be a solution. Preferably, the composition comprises substantially no crystalline or solid phase. Preferably, the composition is not a supersaturated solution. The solvent may comprise water and/or a non-aqueous solvent. Inclusion of a non-aqueous solvent may provide certain additional benefits, such as improving the stability of the composition. Compositions of the invention may be in the form of a semi-solid amorphous liquid. Compositions of the invention may be in the form of a gel at normal temperature and pressure. Alternatively, compositions of the invention may be in a dry form or solid form. For example, compositions may be in the form of a powder.

Compositions of the invention may comprise 10 to 5000 ppm of the enzyme. Compositions of the invention may comprise 15 to 3000 ppm of the enzyme. Compositions of the invention may comprise 20 to 2500 ppm of the enzyme. Compositions of the invention may comprise 25 to 2000 ppm of the enzyme, for example 50 to 1000 ppm of the enzyme. Compositions of the invention may comprise 750 to 2000 ppm of the enzyme. Compositions of the invention may comprise greater than 500 ppm of the enzyme. Compositions of the invention may comprise 250 to 1500 ppm of the enzyme.

The enzyme may be 0.0005% to 0.5% (by weight), 0.001% to 0.2% (by weight), 0.001% to 0.1% (by weight) enzyme, or 0.01% to 0.05% (by weight) of the composition. The enzyme may be 0.001% to 0.01% (by weight) of the composition.

The enzyme activity (for example, the glucose oxidase activity) may range, for example, from 1-400 IU/mg, or 1-300 IU/mg, for example 250-280 IU/mg. The amount of enzyme used is likely to depend on several factors, including the desired use of the composition, the desired level of hydrogen peroxide release, and the desired length of time for hydrogen peroxide release. A suitable amount of enzyme can readily be determined by a person of ordinary skill in the art, if necessary using a well diffusion assay, to determine the extent of hydrogen peroxide release for different amounts of enzyme. The amount of enzyme used may be selected so as to produce a composition for generating antimicrobial activity that is equivalent to a selected phenol standard (for example a 10%, 20%, or 30% phenol standard).

Compositions of the invention may comprise at least 1 unit, and preferably up to 1500 units, of the enzyme per gram of the composition. A “unit” is defined herein as the amount of enzyme (e.g. glucose oxidase) causing the oxidation of 1 micromole of substrate (e.g. glucose) per minute at 25 degrees centigrade at pH 7.0.

Preferably, the enzyme is, or comprises, an oxidoreductase enzyme. Examples of oxidoreductase enzymes include glucose oxidase, hexose oxidase, cholesterol oxidase, galactose oxidase, pyranose oxidase, choline oxidase, pyruvate oxidase, glycollate oxidase, amino acid oxidase, or mannose oxidase.

Preferably, the oxidoreductase enzyme is glucose oxidase and the substrate for the oxidoreductase enzyme is glucose.

In some embodiments, a composition according to the invention comprises more than 15 units, for example at least 30 units, at least 50 units, or at least 100 units, and suitably less than 685 units, for example 100-500 units, of enzyme (e.g. glucose oxidase) per gram of the composition. In other embodiments of the invention, a composition according to the invention comprises at least 500 units, for example 500-1000 units, or 685-1000 units, of enzyme (e.g. glucose oxidase) per gram of the composition.

References herein to “substrate” encompass one or more substrates. For example, in some embodiments, compositions of the invention may comprise a plurality of substrates. In some embodiments, compositions of the invention may comprise only one substrate.

Preferably, the substrate is a purified substrate. The term “purified substrate” is used herein to include a substrate which has been separated from at least some of the impurities originally present when the substrate was obtained or produced. The purified substrate may be obtained from a natural source or may be synthetically produced. The purified substrate may be a processed, extracted, or refined substrate (i.e. a substrate in which impurities or unwanted elements have been removed by processing).

It may not always be necessary or desirable that the purified substrate is at a high level of purity provided that the enzyme is able to convert the substrate to release hydrogen peroxide. In some circumstances, it may be desirable to use a relatively crude substrate. Examples of suitable purity levels include at least 10%, 20%, 30%, 40%, 50%, 60%, 70%, 80%, 90%, 95%, or 99% pure (mass purity). Preferably the purity level is at least 90%. Even more preferably, the purity is at least 99%. However, in some embodiments, it may be desirable that the purified substrate is a medical grade, medical device grade, or pharmaceutical grade substrate.

In particular embodiments, the substrate is, or comprises, sugar. The term “sugar” is used herein to refer to a carbohydrate with the general formula Cₘ(H₂O)ₙ. The purified sugar may be obtained from a natural source (for example a processed, extracted, or refined natural sugar), or be synthetically produced. The sugar may be at least 10%, 20%, 30%, 40%, 50%, 60%, 70%, 80%, 90%, 95%, or 99% pure (mass purity). Preferably the purity level is at least 90%. Even more preferably, the purity is at least 99%. The sugar may be a medical grade, medical device grade, or pharmaceutical grade sugar. The sugar may be a monosaccharide or a disaccharide, preferably a monosaccharide. The sugar may include, for example purified D-glucose, hexose, or D-galactose. For example, the purified sugar may be medical grade, medical device grade, or pharmaceutical grade D-glucose, hexose, or D-galactose. The sugar may be an anhydrous sugar. For example, the glucose may be anhydrous glucose.

Instead of, or in addition to, the substrate, the composition may comprise a precursor substrate. Any disclosure herein which relates to the substrate, such as amounts and purity, may also apply to the precursor substrate.

For compositions of the invention which comprise a precursor-substrate, the composition may comprise one or more enzymes for converting the precursor-substrate to the substrate for the enzyme. However, in some embodiments, the precursor-substrate may not necessarily be converted to the substrate enzymatically. For example, for some precursor substrates, addition of water may be sufficient for conversion. Alternatively or additionally, compositions of the invention may comprise non-enzymatic catalysts.

Compositions which comprise a precursor-substrate may comprise a first enzyme that is able to convert the substrate to release hydrogen peroxide, and a second enzyme that is able to convert the precursor-substrate to the substrate for the first enzyme.

The precursor-substrate is preferably a carbohydrate, such as a polysaccharide, or a sugar e.g. a disaccharide, or sugar derivative. For example, the precursor-substrate may be sucrose, the first enzyme may be glucose oxidase and the second enzyme may be invertase. In another example, the precursor-substrate may be maltose, the first enzyme may be glucose oxidase and the second enzyme may be maltase.

Compositions of the invention which comprise a precursor-substrate may comprise an enzyme (preferably a purified enzyme) that is able to convert the substrate to release hydrogen peroxide, and at least two enzymes (e.g. second and third enzymes, preferably purified enzymes) that are able to convert the precursor-substrate to the substrate for the first enzyme. For example, the precursor-substrate may be starch, the first enzyme may be glucose oxidase and the second and third enzymes may be amylase and maltase. For example, the precursor-substrate may be cellulose, the first enzyme may be glucose oxidase and the second and third enzymes may be cellulose and beta-glucosidase.

Compositions of the invention preferably comprise an additional component in the form of solute. References herein to “solute” encompass one or more solutes. For example, in some embodiments, compositions of the invention may comprise a plurality of solutes. In some embodiments, the composition may comprise only one solute. Preferably, the solute is soluble in water.

The solute may be distinct from the substrate, or in some examples, the substrate may be the same as the solute. For example, the composition may comprise fructose and fructose oxidase: the fructose being both the solute and the substrate for enzyme. In preferred embodiments, the solute is different from the substrate. In one particularly preferred embodiment, the substrate is glucose and the solute is fructose.

The solute is preferably purified, meaning that the solute has been separated from at least some of the impurities originally present when the solute was obtained or produced. The solute may be obtained from a natural source or may be synthetically produced. The solute may be a processed, extracted, or refined substrate (i.e. a solute in which impurities or unwanted elements have been removed by processing). It may not always be necessary or desirable that the solute is at a high level of purity. In some circumstances, it may be desirable to use a relatively crude solute preparation. Examples of suitable purity levels include at least 10%, 20%, 30%, 40%, 50%, 60%, 70%, 80%, 90%, 95%, or 99% pure (mass purity). Preferably, the purity level is at least 90%. More preferably, the purity is at least 99%. However, in some embodiments, it may be desirable that the solute is a medical grade, medical device grade, or pharmaceutical grade solute.

In a preferred embodiment, the enzyme is at least 95% pure, the solute is at least 95% pure and the substrate is at least 95% pure (all with reference to mass purity).

In another preferred embodiment, the enzyme is at least 98% pure, the solute is at least 98% pure and the substrate is at least 98% pure (all with reference to mass purity).

In another preferred embodiment, the enzyme is at least 99% pure, the solute is at least 99% pure and the substrate is at least 99% pure (all with reference to mass purity).

Compositions of the invention, or components of compositions of the invention, may be pharmaceutical grade. The term “pharmaceutical grade” is used herein to refer to include reference to a purity standard for a reagent that has been established by a recognized national or regional pharmacopeia (e.g., the U.S. Pharmacopeia (USP), British Pharmacopeia (BP), National Formulary (NF), European Pharmacopoeia (EP), or Japanese Pharmacopeia (JP)).

The solute may be, or may comprise, a carbohydrate. The solute may be, or may comprise, a polysaccharide. Preferably, the solute is, or comprises, sugar or sugar derivative. More preferably, the solute is, or comprises, a sugar. Suitable sugars include oligosaccharides, disaccharides or monosaccharides. Preferably, the sugar is a disaccharide or a monosaccharide. In particularly preferred embodiments, the sugar is a monosaccharide. Suitable sugars may include fructose, glucose, galactose, sucrose, maltose. In a particularly preferred embodiment, the sugar is fructose.

“Sugar derivative” is used herein to refer to a sugar that has been modified by addition of one or more substituents. The substituent may be something other than a hydroxyl group. Sugar derivatives encompasses amino sugars, acidic sugars, deoxy sugars, sugar alcohols, glycosylamines and sugar phosphates. For example, sugar derivatives may include glucose-6-phosphateglucosamine, glucoronate, gluconate, galactosamine, glucosamine, sialic acid, deoxyribosefucose, rhamnose glucuronic acid, polyols (e.g. sorbitol, erythritol, xylitol, mannitol, lactitol and maltitol) and sucralose.

Compositions of the invention may comprise two or more solutes, as described herein. For example, compositions of the invention may comprise two or more sugars or sugar derivatives. The composition may comprise a maximum of two solutes, e.g. two sugars or sugar derivatives; or a maximum of three solutes, e.g. three sugars or sugar derivatives. For instance, a composition of the invention may comprise glucose, fructose and sucrose. However, in a particularly preferred embodiment of the invention, the composition comprises glucose and fructose and no other mono or disaccharides.

If the solute is distinct from the substrate, the amount of the solute in the composition is preferably greater than the amount of the substrate in the composition.

The solute preferably has a high solubility in water, for example a solubility which is greater than glucose. Glucose has a solubility of 90 g/100 g water at 20° C. and 1 atm. In a preferred embodiment, the solute has a solubility greater than or equal to 100 g/100 g water at 20° C. and 1 atm, in a more preferred embodiment, the solute has a solubility greater than or equal to 200 g/100 g water at 20° C. and 1 atm, in an even more preferred embodiment, the solute has a solubility greater than 300 g/100 g water at 20° C. and 1 atm.

A solute with a high solubility may be advantageous because if the composition of the invention is a solution, it may enable the solution to have a relatively high concentration of solute, which may in turn provide a high osmolarity or osmotic strength. Compositions with a high osmolarity or osmotic strength may assist with the antimicrobial efficacy of the composition because they may reduce the amount of water available for microbes or draw water away from microbes, and may assist in wound healing and wound debridement. A solute with a high solubility may also assist in obtaining a desired viscosity.

Fructose is a particularly preferred solute because it has a solubility of about 375 g/100 g water at 20° C. and 1 atm. Consequently, the solute may be fructose.

Surprisingly, the applicant has found that properties of compositions of the invention can be improved by reducing the concentration of substrate. This is counterintuitive, as many existing hydrogen peroxide-generating compositions have focussed on trying to replicate the levels of the sugars present in honey, which typically have levels of glucose well in excess of 20% by weight. For example, a typical blossom honey has a concentration of glucose above 30% by weight. Synthetic compositions with higher levels of glucose similar to those found in honey are described, for example, in WO 2018/065608 (31% glucose), WO 2008/041218 (38%±5 glucose).

The applicant has appreciated that using higher levels of substrate (e.g. glucose) may result in crystallisation. This is particularly disadvantageous for compositions intended to be applied to wounds because it can result in a product with a gritty feel, and one which lacks homogeneity. Furthermore, during storage, crystallisation can take place which can significantly affect the product's shelf-life. Higher levels of substrate may also make the composition difficult to mix effectively during manufacture.

The applicant has also appreciated that inclusion of high levels of substrates such as glucose in compositions for wound treatment could cause detrimental effects. For instance, Xuan YH, et al. (2014) High-Glucose Inhibits Human Fibroblast Cell Migration in Wound Healing via Repression of bFGF-Regulating JNK Phosphorylation. PLoS ONE 9(9): e108182) indicates that studies in diabetic patients implicate high circulating glucose levels with poor wound healing. In particular, the study demonstrated the poor mobilisations of fibroblasts in a cell culture system in the presence of 30 mM glucose.

Consequently, there may be 20% or less, by weight, of the substrate in compositions of the invention. There may be 15% or less by weight, of the substrate. There may be 10% or less, by weight, of the substrate. There may be less than 10% by weight of the substrate. There may be 7.5% or less, by weight of the substrate, e.g. about 5%, by weight of the substrate.

There may be at least 1% by weight, at least 2% by weight, at least 2.5% by weight of the substrate, or at least 3% by weight of the substrate.

For example, compositions of the invention may comprise at least 1% by weight of the substrate but less than 20% by weight of the substrate. In a more preferred embodiment, compositions of the invention may comprise at least 3% by weight of the substrate, but 15% or less by weight of the substrate, for example 1% to 10% by weight of the substrate. The composition may have 2% to less than 10% by weight of the substrate. The composition may have 2.5% to 7.5% by weight of the substrate. The composition may have 1% to 5% by weight of the substrate.

The solute (e.g. fructose) may be present in an amount of at least 20% by weight, at least 25% by weight, at least 40% by weight, at least 50% by weight, at least 60% by weight, at least 65% by weight, at least 70% by weight, or at least 75% by weight.

Some compositions of the invention may have lower levels of solute. Therefore, in preferred embodiments, the amount of solute (e.g. fructose) in the composition is 60% or less, 50% or less, 45% or less, 40% or less, 35% or less, 30% or less. In one embodiment, there is 20 to 60% by weight of the solute in the composition. In a more preferred embodiment, there is 25 to 55% by weight of the solute in the composition. In another preferred embodiment, there is 35 to 50% by weight of the solute in the composition.

Honey has a high concentration of sugars (e.g. monosaccharides and disaccharides). For instance, blossom honey typically has a total sugar content of nearly 80% by weight. The applicant has appreciated that lower total levels of sugar or sugar derivative may further reduce the risk of crystallisation and the unfavourable properties which result from crystallisation. This, again, is contrary to synthetic compositions of the art which are based on recreating the levels of solutes, such as sugars, which naturally occur in honey. WO 2018/065608, for example, discloses compositions with a total sugar content of 83% by weight, with fructose being present in an amount of 52% by weight. WO 2008/041218, for example, discloses synthetic compositions with a total sugar content of 84.5%, including glucose, fructose, maltose and sucrose.

The solute may contribute to a desirable viscosity of the composition, it may act as a bulking agent and it may act as a water activity moderator, to ensure that the composition maintains a low water activity and very low levels of hydrogen peroxide prior to dilution. In this respect, fructose may be particularly beneficial.

Synthetic hydrogen-peroxide generating compositions are known in the art, of which many are closely based on the levels of various components that are found naturally in honey. For example, WO2008/041218 discloses synthetic compositions with 35±5% fructose and 38±5% glucose. WO2018/065608 discloses compositions 52% fructose and 31% glucose. The levels of sugars in such compositions are very similar to the levels naturally found in honey.

High levels of sugars can provide benefits. A high osmolarity or osmotic strength may assist with the antimicrobial efficacy of the composition because they may reduce the amount of water available for microbes or draw water away from microbes, and may assist in wound healing and wound debridement. High levels of sugars can also enhance viscosity, which can be beneficial for compositions which are intended to be retained on a wound.

However, the applicant has appreciated that although high levels of sugars can provide benefits, they may also cause various disadvantages. Compositions with high sugar concentrations may be unstable and may be prone to crystallisation and separation. High levels of sugar may also cause pain when administered to a wound, resulting from the high osmotic pressure generated. As indicated above, high sugar levels are also implicated in poor wound healing.

Nevertheless, the applicant has appreciated that there may still be benefits in maintaining maintain low levels of sugars. The vascular systems of patients with wounds may be compromised, meaning nutrients may not be able to reach the wound. A composition that provides sugars may thus provide the wound with an efficient energy source, avoiding the breakdown of proteins by bacteria reducing generation of odour-causing compounds such as hydrogen sulphide. Furthermore, the presence of sugars can also contribute to minimising the amount of free water in the composition, thus contributing to stability of the composition and minimising hydrogen peroxide production until it is necessary, upon dilution of the composition. Low levels of sugars may still affect the viscosity of the composition and the osmotic activity of the composition, supporting wound debridement.

In compositions of the invention, the total amount of sugar (e.g. monosaccharides and disaccharides) is preferably less than 20% by weight. Preferably, the total amount of sugar is 10% or less by weight. Preferably the total amount of sugar is 7.5% or less, by weight. In one example, the total amount of sugar in compositions of the invention is 1% to 10% by weight. In one embodiment, the total amount of sugar in the composition is 1 to 7.5% by weight.

According to the invention there is provided a liquid or gel composition, comprising: enzyme that is able to convert a substrate to release hydrogen peroxide; substrate for the enzyme; and polymer, wherein there is less than 20% sugar, by weight, in the composition.

According to the invention there is provided a liquid or gel composition, comprising: enzyme that is able to convert a substrate to release hydrogen peroxide; substrate for the enzyme; and polymer, wherein there is less than 20% sugar, by weight, in the composition, and wherein the composition has a water activity of 0.7 or less, preferably 0.6 or less.

According to the invention there is provided a liquid or gel composition, comprising: enzyme that is able to convert a substrate to release hydrogen peroxide; substrate for the enzyme; and polymer, wherein there is less than 20% sugar, by weight, in the composition, and wherein the composition does not include sufficient free water to allow the enzyme to convert the substrate.

According to the invention there is provided a liquid or gel composition, comprising: enzyme that is able to convert a substrate to release hydrogen peroxide; substrate for the enzyme; and polymer, wherein there is less than 20% sugar, by weight, in the composition, and wherein the composition comprises less than 10 ppm hydrogen peroxide, preferably 6 ppm or less hydrogen peroxide, more preferably 3 ppm or less hydrogen peroxide.

According to the invention there is provided a liquid or gel composition, comprising: enzyme that is able to convert a substrate to release hydrogen peroxide; substrate for the enzyme; and polymer, wherein there is less than 20% sugar, by weight, in the composition, and wherein there is less than 20% water, by weight, in the composition, preferably less than 10% by weight of water in the composition.

According to the invention there is provided a method of preparing a liquid or gel composition, the method comprising, combining: an enzyme that is able to convert a substrate to release hydrogen peroxide; and a substrate for the enzyme; and polymer, wherein the composition is formulated such that there is less than 20% by weight of sugar in the composition.

According to the invention there is provided a method of preparing a liquid or gel composition, the method comprising, combining: an enzyme that is able to convert a substrate to release hydrogen peroxide; and a substrate for the enzyme; and polymer, wherein the composition is formulated such that there is not sufficient free water to allow the enzyme to convert the substrate, and wherein the composition is formulated such that there is less than 20% by weight of sugar in the composition.

According to the invention there is provided a method of preparing a liquid or gel composition, the method comprising, combining: an enzyme that is able to convert a substrate to release hydrogen peroxide; and a substrate for the enzyme; and polymer, wherein the composition is formulated to have a water activity of 0.7 or less, preferably 0.6 or less and wherein the composition is formulated such that there is less than 20% by weight of sugar in the composition.

According to the invention there is provided a method of preparing a liquid or gel composition, the method comprising, combining: an enzyme that is able to convert a substrate to release hydrogen peroxide; and a substrate for the enzyme; and polymer, wherein the composition is formulated such that there is less than 20% by weight of water in the composition, preferably less than 10% by weight of water in the composition, and wherein the composition is formulated such that there is less than 20% by weight of sugar in the composition.

According to the invention there is provided a method of preparing a liquid or gel composition, the method comprising, combining: an enzyme that is able to convert a substrate to release hydrogen peroxide; and a substrate for the enzyme; and polymer, wherein the composition is formulated such that there is less than 10 ppm of hydrogen peroxide in the composition, preferably less than 6 ppm hydrogen peroxide in the composition, and wherein the composition is formulated such that there is less than 20% by weight of sugar in the composition.

Compositions of the invention may contain less than 10% by weight of solute (e.g. fructose) and less than 10% by weight of substrate (e.g. glucose). Compositions of the invention may contain 1-10% by weight of solute (e.g. fructose) and 1-10% by weight of substrate (e.g. glucose). Compositions of the invention may contain 1-5% by weight of solute (e.g. fructose) and 1-5% by weight of substrate (e.g. glucose).

Compositions of the invention may comprises at least two sugars or sugar derivatives (e.g. including glucose and fructose). The composition may comprise a maximum of two sugars or sugar derivatives (e.g. only glucose and fructose). Compositions of the invention may comprise only one sugar, such as glucose.

Compositions of the invention may not comprise fructose. Compositions of the invention may not comprise sucrose. Compositions of the invention may not comprise any disaccharides.

Compositions of the invention may comprise a buffer. An example of a suitable buffer is a citric acid/NaOH buffer, or TRIS/HCl buffer. The buffer may be monosodium and/or disodium phosphate.

The buffer which is included in compositions of the invention may be 0.05 to 0.3 M, such as 0.1 to 0.2 M. The buffer may be pre-formed and then added with the other components to form compositions of the invention. For example, 1 to 20% or 1 to 10% by weight of the preformed buffer may be added to form compositions of the invention.

In compositions of the invention the buffer may be present in a concentration of 2 to 200 μM, such as 5 to 100 μM. The concentration of buffer may be 10 μM, for example.

Patient wounds can be sensitive and the introduction of an acidic material directly into the wound will cause discomfort. Consequently, a more neutral pH may be preferred. The composition may have a pH of 6 to 8, preferably 6.5 to 7.5, or more preferably about 7.0.

Glucose oxidase's optimal activity is around pH 5.5 to 6.0. However there is also a feedback loop in the reaction such that as gluconic acid is produced from the breakdown of glucose it may begin to inhibit glucose oxidase and the production of hydrogen peroxide may decline. Starting at a more neutral pH may assist in preventing the enzyme initiating prematurely.

The choice of pH and buffer may also allow the neutralisation of gluconic acid, so that upon dilution of the product which initiates hydrogen peroxide production, the level of hydrogen peroxide production can be sustained over at least 72 hours. If the pH of the composition was lower, the production of the gluconic acid may curtail the enzyme activity far sooner, reducing the effectiveness of the product.

Compositions of the invention may have a viscosity (dynamic viscosity), of at least 10000 mPas at 20° C. and 1 atm. Compositions of the invention may have a viscosity of 10000 to 20000 mPas at 20° C. and 1 atm, more preferably 12000 to 15000 mPas at 20° C. and 1 atm. The applicant has appreciated that desirable viscosities may be achieved with lower levels of sugars or sugar derivatives. This can be achieved, for example, by including thickeners, viscosity enhancers or gelling agents

Some embodiments may have a low viscosity, for example if they are intended for spraying. Consequently, the viscosity of the composition may be less than 10000 mPas at 20° C. and 1 atm, preferably less than 1000 mPas at 20° C. and 1 atm. Compositions of the invention may have a viscosity of 50 to 200 mPas at 20° C. and 1 atm, such as 70 to 120 mPas at 20° C. and 1 atm. Compositions of the invention may be sprayable.

Compositions of the invention preferably comprise a polymer. The polymer in the composition may be any medically acceptable polymer, such as any Food and Drug Administration-approved (FDA-approved) polymer.

In some embodiments, the polymer may be a synthetic polymer. In some embodiments, the polymer is a natural polymer, such as a polysaccharide.

Preferably, the polymer is water soluble. The polymer may be soluble in an organic, or non-aqueous, solvent. The polymer may be soluble in a mixture of an aqueous and non-aqueous solvent. The polymer may be biodegradable or bioerodable. The polymer may be a co-polymer.

In a preferred embodiment, the polymer is a water-soluble synthetic polymer. In preferred embodiments, the polymer is selected from polyethylene oxide (or polyethylene glycol), polyvinyl alcohol and polyvinylpyrrolidone. Other polymers may include poly(lactic-co-glycolic acid), polyglycolic acid, polylactic acid, polycaprolactone, polymeric surfactants, polyacrylic acid, polyacrylamide, N-(2-hydroxypropyl) methacrylamide (HPMA), polyoxazolines, polyphosphates or polyphosphazenes. Another suitable polymer may be phosphino-carboxylic acid (PCA). In a preferred example, the polymer is, or comprises polyethylene glycol (PEG). Further polymers may include polysaccharides such as cellulose (which includes derivatives such as hydroxypropyl methyl cellulose and hydroxypropyl cellulose), alginate, gelatin or cyclodextrins. Suitable polymers may also include chitosan or hyaluronic acid.

In addition to affecting viscosity of the composition, polymers can contribute to the osmotic pressure of the composition, assisting wound debridement. In addition, polymers such as polyethylene glycol, may have antimicrobial properties.

Preferably, compositions of the invention contain polymer in an amount greater than 50% by weight. Compositions, of the invention contain polymer in an amount of at least 60% by weight. For example, compositions of the invention may contain polymer in an amount of greater than 50% by weight, to 80% by weight. compositions of the invention may contain polymer in an amount of greater than 50% by weight, up to 70% by weight.

In other preferred compositions of the invention, polymer may be present in an amount of at least 30% by weight. Polymer may be present in compositions of the invention in an amount of 70% or less by weight. So, in some preferred embodiments, polymer may be present in an amount of 30% to 70%, by weight.

Use of high concentrations of polymer can afford desirable physical characteristics to the composition, such as a high viscosity, without having to rely on a high sugar content.

Furthermore, the use of high concentrations of polymers can reduce the stickiness of the composition, making it more amenable for patient application.

According to the invention, there is provided a liquid or gel composition comprising enzyme that is able to convert a substrate to release hydrogen peroxide; substrate for the enzyme; and polymer, wherein the polymer is present in an amount greater than 50% by weight of the composition.

According to the invention, there is provided a liquid or gel composition comprising enzyme that is able to convert a substrate to release hydrogen peroxide; substrate for the enzyme; and polymer, wherein the polymer is present in an amount greater than 50% by weight of the composition, and wherein the composition has a water activity of 0.7 or less, preferably 0.6 or less.

According to the invention, there is provided a liquid or gel composition comprising enzyme that is able to convert a substrate to release hydrogen peroxide; substrate for the enzyme; and polymer, wherein the polymer is present in an amount greater than 50% by weight of the composition, and wherein the composition does not include sufficient free water to allow the enzyme to convert the substrate.

According to the invention, there is provided a liquid or gel composition comprising enzyme that is able to convert a substrate to release hydrogen peroxide; substrate for the enzyme; and polymer, wherein the polymer is present in an amount greater than 50% by weight of the composition, and wherein the composition comprises less than 10 ppm hydrogen peroxide, preferably 6 ppm or less hydrogen peroxide, more preferably 3 ppm or less hydrogen peroxide.

According to the invention, there is provided a liquid or gel composition comprising enzyme that is able to convert a substrate to release hydrogen peroxide; substrate for the enzyme; and polymer, wherein the polymer is present in an amount greater than 50% by weight of the composition, and wherein there is less than 20% water, by weight, in the composition, preferably less than 10% water, by weight, in the composition.

According to the invention there is provided a method of preparing a liquid or gel composition, the method comprising, combining: an enzyme that is able to convert a substrate to release hydrogen peroxide; a substrate for the enzyme; and polymer, wherein the polymer is added such that it is greater than 50% by weight of the composition.

According to the invention there is provided a method of preparing a liquid or gel composition, the method comprising, combining: an enzyme that is able to convert a substrate to release hydrogen peroxide; a substrate for the enzyme; and polymer, wherein the composition is formulated to have a water activity of 0.7 or less, preferably 0.6 or less, and wherein the polymer is added such that it is greater than 50% by weight of the composition.

According to the invention there is provided a method of preparing a liquid or gel composition, the method comprising, combining: an enzyme that is able to convert a substrate to release hydrogen peroxide; a substrate for the enzyme; and polymer, wherein the composition is formulated such that it does not include sufficient free water to allow the enzyme to convert the substrate, and wherein the polymer is added such that it is greater than 50% by weight of the composition.

According to the invention there is provided a method of preparing a liquid or gel composition, the method comprising, combining: an enzyme that is able to convert a substrate to release hydrogen peroxide; a substrate for the enzyme; and polymer, wherein the composition is formulated such that there is less than 20% by weight of water in the composition, preferably less than 10% by weight of water in the composition, and wherein the polymer is added such that it is greater than 50% by weight of the composition.

According to the invention there is provided a method of preparing a liquid or gel composition, the method comprising, combining: an enzyme that is able to convert a substrate to release hydrogen peroxide; and a substrate for the enzyme; and polymer, wherein the composition is formulated such that there is less than 10 ppm of hydrogen peroxide in the composition, preferably less than 6 ppm hydrogen peroxide in the composition, and wherein the polymer is added such that it is greater than 50% by weight of the composition.

It is advantageous for compositions of the invention to have rheological properties which permit them to be readily applied to a wound by, for example, easy extrusion from packaging, yet remain in effective contact with a wound for the duration of treatment. Although compositions which contain high levels of sugars (e.g. levels similar to those found in honey) can create viscous compositions which can be applied effectively to a wound, the applicant has found that such compositions may be prone to becoming less viscous as they warm up to body temperature, which can decrease it efficacy. By using thickeners or viscosity enhancers and lower levels of sugars or sugar derivatives, the applicant has found that such compositions are more effective at remaining in position on a wound. Furthermore, the lower levels of sugars can reduce the risk of crystallisation, as described above.

Compositions may comprise a mixture of polymers. For example, the applicant has found that viscosity can be fine-tuned for various applications by blending polymers of varying molecular weight, such as polyethylene glycol polymers of varying molecular weight. If a less viscous composition is desired, such as a composition intended for spraying, lower molecular weight polymers, such as PEG 300, may be preferred. If a more viscous composition is desired, higher molecular weight polymers, such as PEG 4000, may be preferred. So, in some embodiments, the polymer comprises a first polymer and a second polymer, wherein the first polymer has a lower molecular weight than the second polymer. The first polymer may have a molecular weight less than 1000, and the second polymer may have a molecular weight of greater than 1000. The first polymer may have a molecular weight less than 500. The second polymer may have a molecular weight greater than 3000. There may be a third polymer of intermediate molecular weight to the first and second polymer. The third polymer may have a molecular weight of 1000 to 3000.

In some embodiments, higher molecular weight polymers (such as polyethylene glycols) may be preferred. It has been postulated that in certain circumstances, lower molecular weight polymers may ‘trap’ water, enzyme and substrate which may delay release of hydrogen peroxide when diluted. Consequently, in some embodiments, compositions may not comprise polymers with a molecular weight less than 500. In some embodiments, only polymers with a molecular weight of 600 or higher may be employed (see, for instance, Example 9).

Viscosity enhancers such as hydrocolloids may be included in compositions of the invention. Hydrocolloids are a heterogeneous group of hydrophilic, long-chain polymers (polysaccharides or proteins) characterised by their ability to form viscous dispersions and/or gels when dispersed in water (Saha and Bhattacharya, J Food Sci Technol, 2010, 47(6):587-597). The extent of thickening varies with the type and nature of the hydrocolloid. Some provide low viscosities at a fairly high concentration, but most provide a high viscosity at a concentration below 1%. The viscosity of hydrocolloid dispersions arises predominantly from non-specific entanglement of conformationally disordered polymer chains. Hydrocolloids that can be used as thickening agents (referred to herein as hydrocolloid thickeners) include starch, modified starch, xanthan, galactomannans (such as guar gum, locust bean gum, and tara gum), gum Arabic or acacia gum, gum karaya, gum tragacanth, konjac maanan, and cellulose derivatives such as carboxymethyl cellulose, methyl cellulose, and hydroxypropylmethyl cellulose.

Some hydrocolloids are able to form gels, consisting of polymer molecules cross-linked to form an interconnected molecular network immersed in a liquid medium. A rheological definition of a gel is a viscoelastic system with a ‘storage modulus’ (G′) larger than the ‘loss modulus’ (G″) (de Vries 2004, Gums and stabilizers for the food industry, vol 12. RSC Publ, Oxford, pp 22-30). Hydrocolloids form gels by physical association of their polymer chains through hydrogen bonding, hydrophobic association, and cation-mediated cross-linking. Gelling-type hydrocolloids (or hydrocolloid gelling agents) include alginate, pectin, carrageenan, gelatin, gellan, agar, modified starch, methyl cellulose and hydroxypropylmethyl cellulose.

Gelation of hydrocolloids can occur by different mechanisms: ionotropic gelation, cold-set gelation and heat-set gelation (Burey et al. 2008, Crit Rev Food Sci Nutr 48:361-377). lonotropic gelation occurs via cross-linking of hydrocolloid chains with ions, typically a cation-mediated gelation process of negatively-charged polysaccharides. Examples of hydrocolloids that can form gels by ionotropic gelation include alginate, carrageenan and pectin. lonotropic gelation can be carried out by either diffusion setting or internal gelation. In cold-set gelation, hydrocolloid powders are dissolved in warm/boiling water to form a dispersion which forms a gel on cooling. Agar and gelatin form gels by this mechanism. Heat-set gels require the application of heat to gel (for example, curdlan, konjac glucomannan, methyl cellulose, starch and globular proteins).

In particular embodiments, the hydrocolloid is, or comprises, a polysaccharide or a protein. The hydrocolloid may be a hydrocolloid thickener, such as starch, modified starch, xanthan, a galactomannan (such as guar gum, locust bean gum, and tara gum), gum Arabic or acacia gum, gum karaya, gum tragacanth, konjac maanan, or a cellulose derivative, such as carboxymethyl cellulose, methyl cellulose, or hydroxypropylmethyl cellulose.

In other embodiments, the hydrocolloid is, or comprises a cross-linked hydrocolloid, for example a cross-linked polysaccharide, such as cross-linked alginate, pectin, carrageenan, gelatin, gellan, agar, agarose, modified starch, or a cellulose derivative, such as methyl cellulose or hydroxypropylmethyl cellulose.

The hydrocolloid may be cross-linked by any suitable method, for example including the methods for gelation of hydrocolloids described above: ionotropic gelation, cold-set gelation and heat-set gelation. In particular embodiments, molecules of the hydrocolloid are cross-linked by cations (for example calcium ions) as a result of ionotropic gelation of a hydrocolloid gelling agent. Examples of hydrocolloid cross-linked by cations that may be present in a composition of the invention include alginate, carrageenan or pectin.

In particular embodiments, a composition of the invention includes cross-linked alginate, for example alginate cross-linked by calcium ions. Alginate can form gels without prior heating because sodium alginate is soluble in cold water.

Cross-linked alginate may be formed from sodium alginate and calcium ions (for example, provided by calcium chloride). In some embodiments, water may be used as solvent to dissociate the calcium ions. However, since this could potentially activate production of hydrogen peroxide by the enzyme and the substance that includes a substrate for the enzyme, and limit the stability of the composition, it may be preferred to use a non-aqueous solvent to dissociate the calcium ions, such as ethanol or acetic acid.

Compositions of the invention preferably contain substantially no catalase. Compositions of the invention preferably contain essentially no catalase. In some embodiments, compositions of the invention may comprise catalase.

Compositions of the invention preferably contain substantially no peroxidase. Compositions of the invention preferably contain essentially no peroxidase.

Compositions of the invention preferably contain substantially no lactoferrin. Compositions of the invention preferably contain essentially no lactoferrin.

Compositions of the invention preferably do not comprise an unrefined substance. The term “unrefined” is used herein to refer to substances that have not been processed into a pure form. Unrefined substances include substances that may have been concentrated, for example by drying or boiling.

Compositions of the invention preferably do not include one or more substrates from a natural source (termed herein a “natural substance”). Examples of natural substances include substances from a plant source, including from sap, roots, nectar, flowers, seeds, fruit, leaves, or shoots.

Preferably, compositions of the invention do not comprise an unrefined natural substance. Compositions of the invention preferably do not comprise honey.

Compositions of the invention may comprise additional components that may aid in the stability of the composition, such as the stability of the enzyme. A composition of the invention may thus comprise a protein-stabilising excipient.

A composition of the invention may comprise non-aqueous solvent, such as an organic solvent, e.g. a polar organic solvent. In compositions of the invention that comprise non-aqueous solvent, the non-aqueous solvent may comprise ethanol, dimethyl sulphoxide, glycerol (or glycerine or glycerin, as it is otherwise known), ethylene glycol or propylene glycol. Preferably, the non-aqueous solvent is or comprises a polyol. An example of a particularly suitable polyol is glycerol. The applicant has found that non-aqueous solvents (e.g. polyols such as glycerol) can be effective at providing a stable and effective composition.

Non-aqueous solvents, such as glycerol, may contribute to the osmotic pressure of the composition, thus assisting in wound debridement. Such non-aqueous solvents may also have antimicrobial properties. For example, glycerol can be an effective antimicrobial against gram positive organisms.

Non-aqueous solvent may comprise only one non-aqueous solvent, or it may comprise a plurality of non-aqueous solvents.

Compositions of the invention preferably comprise a non-aqueous solvent and water.

In preferred embodiments, the compositions may comprise at least 2% by weight of the non-aqueous solvent, at least 5% by weight of the non-aqueous solvent, at least 10% by weight of the non-aqueous solvent, at least 20% by weight of the non-aqueous solvent at least 30% by weight of the non-aqueous solvent or at least 40% by weight of the non-aqueous solvent. The non-aqueous solvent may be present in an amount of 50% by weight or less, 40% by weight or less, 30% by weight or less, or 20% by weight or less.

For example, there may be 2% to 50% by weight non-aqueous solvent in the composition. There may be 5% to 40% by weight non-aqueous solvent in the composition. There may be 15 to 30% by weight non-aqueous solvent in the composition. There may be 20% to 40% by weight non-aqueous solvent in the composition.

In some embodiments, the amount of non-aqueous solvent in the composition may be 10% to 75%, by weight, preferably 20% to 60% by weight, more preferably 25% to 60% by weight.

In some embodiments, the non-aqueous solvent may be in an amount greater than 50%, by weight. For example, the non-aqueous solvent may be in an amount of at least 60% by weight, at least 70%, by weight, or at least 80% by weight. The inventors have appreciated that if a composition of the invention is intended for application to the upper respiratory tract, such as by administration orally or nasally, the presence of a polymer (such as PEG) may have a bitter taste. If at least some polymer is substituted with non-aqueous solvent (e.g. glycerol), the taste may be less bitter.

In some embodiments, compositions of the invention may comprise substantially no polymer. For example, some compositions with high amounts of non-aqueous solvent may not comprise any polymer.

In some embodiments, polymer may be present in a composition of the invention in an amount of at 30% by weight and non-aqueous solvent may be present in an amount of at least 20% by weight.

In some embodiments, polymer may be present in a composition of the invention in an amount of at 35% by weight and non-aqueous solvent may be present in an amount of at least 40% by weight.

In some embodiments, polymer may be present in a composition of the invention in an amount of at least 50% by weight and non-aqueous solvent may be present in an amount of at least 20% by weight.

In some embodiments, polymer may be present in a composition of the invention in an amount of 30% to 50% by weight and non-aqueous solvent may be present in an amount of 40% to 60% by weight.

In some embodiments, polymer may be present in a composition of the invention in an amount of 50% to 70% by weight and non-aqueous solvent may be present in an amount of 20% to 40% by weight.

In some embodiments, polymer may be present in a composition of the invention in an amount of 30% to 70% by weight and non-aqueous solvent may be present in an amount of 20% to 60% by weight.

According to the invention, there is provided a liquid or gel composition comprising enzyme that is able to convert a substrate to release hydrogen peroxide; substrate for the enzyme; and non-aqueous solvent, wherein the non-aqueous solvent is present in an amount greater than 50% by weight of the composition, optionally wherein: i) there is less than 20%, by weight, of sugar in the composition; ii) the substrate is 5% or less, by weight, of the composition; and/or iii) the composition does not comprise honey.

According to the invention, there is provided a liquid or gel composition comprising enzyme that is able to convert a substrate to release hydrogen peroxide; substrate for the enzyme; and non-aqueous solvent, wherein the non-aqueous solvent is present in an amount greater than 50% by weight of the composition, and wherein the composition has a water activity of 0.7 or less, preferably 0.6 or less, optionally wherein: i) there is less than 20%, by weight, of sugar in the composition; ii) the substrate is 5% or less, by weight, of the composition; and/or iii) the composition does not comprise honey.

According to the invention, there is provided a liquid or gel composition comprising enzyme that is able to convert a substrate to release hydrogen peroxide; substrate for the enzyme; and non-aqueous solvent, wherein the non-aqueous solvent is present in an amount greater than 50% by weight of the composition, and wherein the composition does not include sufficient free water to allow the enzyme to convert the substrate, optionally wherein: i) there is less than 20%, by weight, of sugar in the composition; ii) the substrate is 5% or less, by weight, of the composition; and/or iii) the composition does not comprise honey.

According to the invention, there is provided a liquid or gel composition comprising enzyme that is able to convert a substrate to release hydrogen peroxide; substrate for the enzyme; and non-aqueous solvent, wherein the non-aqueous solvent is present in an amount greater than 50% by weight of the composition, and wherein the composition comprises less than 10 ppm hydrogen peroxide, preferably 6 ppm or less hydrogen peroxide, more preferably 3 ppm or less hydrogen peroxide, optionally wherein: i) there is less than 20%, by weight, of sugar in the composition; ii) the substrate is 5% or less, by weight, of the composition; and/or iii) the composition does not comprise honey.

According to the invention, there is provided a liquid or gel composition comprising enzyme that is able to convert a substrate to release hydrogen peroxide; substrate for the enzyme; and non-aqueous solvent, wherein the non-aqueous solvent is present in an amount greater than 50% by weight of the composition, and wherein there is less than 20% water, by weight, in the composition, preferably less than 10% water, by weight, in the composition, optionally wherein: i) there is less than 20%, by weight, of sugar in the composition; ii) the substrate is 5% or less, by weight, of the composition; and/or iii) the composition does not comprise honey.

According to the invention there is provided a method of preparing a liquid or gel composition, the method comprising, combining: an enzyme that is able to convert a substrate to release hydrogen peroxide; a substrate for the enzyme; and non-aqueous solvent, wherein the non-aqueous solvent is added such that it is greater than 50% by weight of the composition, optionally wherein the composition is formulated such that: i) there is less than 20%, by weight, of sugar in the composition; ii) the substrate is 5% or less, by weight, of the composition; and/or iii) the composition does not comprise honey.

According to the invention there is provided a method of preparing a liquid or gel composition, the method comprising, combining: an enzyme that is able to convert a substrate to release hydrogen peroxide; a substrate for the enzyme; and non-aqueous solvent, wherein the composition is formulated to have a water activity of 0.7 or less, preferably 0.6 or less, and wherein the non-aqueous solvent is added such that it is greater than 50% by weight of the composition, optionally wherein the composition is formulated such that: i) there is less than 20%, by weight, of sugar in the composition; ii) the substrate is 5% or less, by weight, of the composition; and/or iii) the composition does not comprise honey.

According to the invention there is provided a method of preparing a liquid or gel composition, the method comprising, combining: an enzyme that is able to convert a substrate to release hydrogen peroxide; a substrate for the enzyme; and non-aqueous solvent, wherein the composition is formulated such that it does not include sufficient free water to allow the enzyme to convert the substrate, and wherein the non-aqueous solvent is added such that it is greater than 50% by weight of the composition, optionally wherein the composition is formulated such that: i) there is less than 20%, by weight, of sugar in the composition; ii) the substrate is 5% or less, by weight, of the composition; and/or iii) the composition does not comprise honey.

According to the invention there is provided a method of preparing a liquid or gel composition, the method comprising, combining: an enzyme that is able to convert a substrate to release hydrogen peroxide; a substrate for the enzyme; and non-aqueous solvent, wherein the composition is formulated such that there is less than 20% by weight of water in the composition, preferably less than 10% by weight of water in the composition, and wherein the non-aqueous solvent is added such that it is greater than 50% by weight of the composition, optionally wherein the composition is formulated such that: i) there is less than 20%, by weight, of sugar in the composition; ii) the substrate is 5% or less, by weight, of the composition; and/or iii) the composition does not comprise honey.

According to the invention there is provided a method of preparing a liquid or gel composition, the method comprising, combining: an enzyme that is able to convert a substrate to release hydrogen peroxide; a substrate for the enzyme; and non-aqueous solvent, wherein the composition is formulated such that there is less than 10 ppm of hydrogen peroxide in the composition, preferably less than 6 ppm hydrogen peroxide in the composition, and wherein the non-aqueous solvent is added such that it is greater than 50% by weight of the composition, optionally wherein the composition is formulated such that: i) there is less than 20%, by weight, of sugar in the composition; ii) the substrate is 5% or less, by weight, of the composition; and/or iii) the composition does not comprise honey.

In some embodiments, compositions of the invention may comprise hydrogen peroxide scavenger or free radical scavenger. For example, compositions of the invention may comprise antioxidant. Antioxidant may consist of one antioxidant or may consist of a plurality of antioxidants. Suitable antioxidants include ascorbic acid, tocopherol or ascorbyl palmitate. Ascorbic acid may be preferred. The applicant has found that antioxidants, such as ascorbic acid, may be particularly effective at reducing the amount of hydrogen peroxide in the composition prior to dilution, and resisting reductions in enzyme activity during storage. Antioxidant may be present in an amount of at least 0.01% by weight or at least 0.1% by weight. Antioxidant may be present in an amount of 2% by weight, or less, 1% by weight, or less, or 0.5% by weight, or less. For example, in some embodiments, antioxidant is present in an amount of 0.1 to 0.5% by weight, such as 0.25% by weight.

Ascorbic acid may also have microbiocidal properties. It may alter cell wall permeability, it may interact with hydrogen peroxide to produce free radicals, and it may inhibit the production of extracellular polymeric substances which confer some of the biofilms to chemical attack. Furthermore, wound healing is delayed without sufficient ascorbic acid. Ascorbic acid may be important for all stages of wound healing such as neutrophil clearance and collagen production.

The concentration of scavenger (e.g. antioxidant) in compositions of the invention may be from 0.01% by weight to 1% by weight. Preferably, the antioxidant is present in an amount of 0.05% by weight to 0.5% by weight. The antioxidant concentration may be less than 0.3% by weight of the composition, for example from 0.1 to 0.25% by weight.

More preferably, the antioxidant is present in an amount of at least 0.1%. The antioxidant may be present in an amount less than 0.5%, such as 0.25%, because higher levels of antioxidant may have the potential to suppress hydrogen peroxide production following dilution. For example, it may suppress the initial burst of hydrogen peroxide production following dilution.

In some compositions of the invention, there may be no hydrogen peroxide scavenger or free radical scavenger, such as an antioxidant. In some compositions, the presence of a scavenger, such as an antioxidant, may reduce effective release of

[symbol]

In some compositions of the invention, there is substantially no oil or other lipophilic phase. Compositions of the invention preferably do not comprise an emulsion.

Compositions of the invention are preferably sterile. Compositions of the invention may be sterilised by any suitable means. Preferably compositions of the invention have been sterilised by irradiation. Irradiation may be achieved by gamma, electron beam or X-ray. The Applicant has found that compositions can retain glucose oxidase activity (and, therefore, the ability to release hydrogen peroxide on dilution) following sterilisation by exposure to gamma irradiation or electron beam irradiation. A suitable level of gamma irradiation is 10-70 kGy, preferably 25-70 kGy, more preferably 35-70 kGy. Alternatively, compositions of the invention may be sterilised by electron beam irradiation. A suitable level or dose of irradiation (e.g. electron beam irradiation) may be 10-100 kGy, preferably 30-80 kGy, more preferably 50-80kGy. The dose may be greater than 35 kGy. The dose may be less than 80 kGy, for example 75 kGy or less. The dose may be 25-50 kGy.

There is also provided according to the invention a method of sterilising a composition of the invention. Preferably, the method comprises exposing the composition to irradiation, preferably gamma irradiation or electron beam irradiation.

Since ozone has not been authorised by the US FDA for sterilisation of honey-based products for use in wound healing, compositions according to the invention preferably have not been sterilized by ozonation, and do not include ozone, or any components that have been subjected to sterilisation by ozonation.

Compositions of the invention may be in a container or sachet. The container may assist in maintaining the sterility of the composition. Preferably, the container is sealed or airtight. The container may have a removable and/or replaceable cap or seal. The container is preferably opaque. The composition may be contained within a syringe or a tube. For example, the composition may be contained within a high-density polyethylene/low-density polyethylene (HDPE/LDPE) tube or in polyester-aluminium-polyethylene (PET/AI/PE) sachet.

A composition of the invention may be provided with a dressing material. The dressing material may be coated with the composition. Suitable dressings materials include gauzes, bandages, tissues, films, gels, foams, hydrocolloids, alginates, hydrogels, or polysaccharide pastes, granules, beads or tulle. It may comprise carboxymethylcellulose. The composition may be present together with a wound-dressing matrix, such as a collagen or collagen-glycosaminoglycan matrix. The dressing may be a tulle dressing. Compositions in combination with a dressing are preferably sterile, and may be sterilised using irradiation, e.g. gamma irradiation or electron beam irradiation.

Compositions of the invention can be used to treat any microbial infection that can be treated by hydrogen peroxide. Examples include infection caused by gram positive bacteria, gram negative bacteria, acid-fast bacteria, viruses, yeasts, parasitic or pathogenic micro-organisms or fungi. For example, infections caused by the following micro-organisms may be treated: Escherichia coli, Staphylococcus aureus, Pseudomonas aeruginosa, Candida albicans, Propionibacterium acnes, Staphylococcus aureus, Staphylococcus epidermidis, Staphylococcus saprophytics, Beta haemolytic Streptococci Group A or B, Campylobacter coli, Campylobacter jejuni, Methicillin Resistant Staphylococcus Aureus (MRSA), Methicillin Sensitive Staphylococcus Aureus (MSSA), Botrytis cinerea, Mycobacterium tuberculosis, Cryptosporidium, Plasmodium, Streptococcus pyogenes, Streptococcus zooepidemicus and Toxoplasma.

There is further provided according to the invention a composition of the invention for use in the prevention or treatment of a microbial infection, for example a microbial infection that comprises a biofilm, or a microbe that is capable of forming a biofilm. So, there may be provided a composition of the invention for use in the prevention or treatment of a microbial infection that comprises a biofilm or a microbe that is capable of forming a biofilm. The biofilm may comprise bacteria, fungi and/or viruses.

There is also provided according to the invention use of a composition of the invention in the manufacture of a medicament for the prevention or treatment of a microbial infection, for example a microbial infection that comprises a biofilm, or a microbe that is capable of forming a biofilm.

The invention also provides a method of preventing or treating a microbial infection, for example a microbial infection that comprises a biofilm, or a microbe that is capable of forming a biofilm, wherein the method comprises administering an effective amount of a composition of the invention to a site of the infection.

According to the invention there is also provided use of a composition of the invention to prevent or inhibit microbial growth.

There is also provided according to the invention a composition of the invention for use as a medicament.

Compositions of the invention may be used to treat animals. Compositions of the invention may be used to treat humans.

There is further provided according to the invention a composition of the invention for the prevention, treatment, or amelioration of a microbial infection.

The invention also provides use of a composition of the invention in the manufacture of a medicament for the prevention, treatment, or amelioration of a microbial infection.

There is further provided according to the invention a method of preventing, treating, or ameliorating a microbial infection, which comprises administering a composition of the invention to a subject in need of such prevention, treatment or amelioration. The subject may be a human or animal subject. Compositions of the invention may be topically administered.

There may be provided compositions of the invention for use in the treatment of a microbial infection that comprises a biofilm.

There is also provided according to the invention a device for delivering a composition to a patient, the device comprising a composition of the invention.

The device may be a spraying or atomising device, such as a pump-action spray or an aerosol spray. The device may be an inhaler, for example, a metered-dose inhaler, a dry powder inhaler, a nebulizer, for delivery of the composition into the lungs, or a nasal inhaler. The spraying device is preferably manufactured from a plastics material, such as made of high density polyethylene (HDPE).The device may be for external use, such as for applying the composition to a patient's skin.

The device may be for internal application to a patient. For example, the device may be a spray, inhaler or nebuliser for administering the composition to the patient's respiratory tract. In one example, the device is a nasal spray or nasal atomiser including a nasal spray actuator designed to apply the composition to a patient's nostril. The device may be a throat spray designed to apply the composition to a patient's throat. Compositions of the invention may thus be used to treat pulmonary infections, such as pulmonary viral infections. Compositions of the invention may be used to treat or prevent viral infections, such as COVID-19. A nasal or throat atomiser device may be manufactured from a plastics material. For example, it may include polypropylene, polyoxymethylene and/or low density polyethylene (LDPE).

The device may be a douche. The device may be a device for injecting the composition into the patient. The device may be a syringe. Compositions of the invention may be for prophylactic applications.

According to a preferred aspect of the invention, a composition of the invention may be used in a method of wound care, including the treatment of a wound, or the treatment or management of wound sepsis.

The wound may be an acute wound, chronic wound, surgical wound (for example, a Caesarean wound), chronic burn, or an acute burn. A composition of the invention may be used in the prophylactic prevention of wound sepsis. If a storage-stable composition of the invention is used, it will be appreciated that this may be diluted by liquid present at the wound site, which thereby leads to the release of hydrogen peroxide by the diluted composition.

There is also provided according to the invention a composition of the invention for treatment of a wound. There is also provided a method of treating a wound, which comprises administering a composition of the invention to a subject in need of such treatment. There is also provided a use of a composition of the invention in the manufacture of a medicament for the treatment of a wound.

Compositions of the invention may be used to treat chronic wounds or wounds that are critically colonized. The term “critically colonized” is often used to refer to a wound that has reached a critical point at which bacteria begin to negatively affect the wound and begin to elicit signs of their presence. A critically colonized wound may indicate the presence of a biofilm. A bacterial load of greater than 10 5 organisms/gram of tissue is often accepted as impeding wound healing (Siddiqui AR, Bernstein JM (2010) Chronic wound infection: Facts and controversies. Clinics in Dermatology 28: 519-26; Edmonds, M., & Foster, A. (2004). The use of antibiotics in the diabetic foot. Am J Surg, 187(5A), 25S-28S. Consequently, compositions of the invention may be used to treat wounds that have a bacterial load of greater than 10⁵ organisms/gram of tissue.

Compositions of the invention may be administered to a patient, such as placed on the wound of a patient, for a period of at least 24 hours or 48 hours or, more preferably, 72 hours.

There is further provided according to the invention use of a composition of the invention in the manufacture of a medicament for treatment of a wound.

There is also provided according to the invention a method of treating inflammation, which comprises administering a composition of the invention to a site of inflammation.

There is also provided according to the invention a composition of the invention for treatment of inflammation.

There is further provided according to the invention use of a composition of the invention in the manufacture of a medicament for treatment of inflammation.

There is also provided according to the invention a method of stimulating tissue growth, which comprises administering a composition of the invention to a site in need of such stimulation.

There is also provided according to the invention a composition of the invention for stimulating tissue growth.

There is further provided according to the invention use of a composition of the invention in the manufacture of a medicament for stimulating tissue growth.

There is also provided according to the invention a method of debriding a wound, which comprises administering a composition of the invention to a wound in need of debridement.

There is also provided according to the invention a composition of the invention for debriding a wound.

There is further provided according to the invention use of a composition of the invention in the manufacture of a medicament for debriding a wound.

There is also provided according to the invention a method of deodorising a wound, which comprises administering a composition of the invention to a wound in need of deodorising.

There is also provided according to the invention a composition of the invention for deodorising a wound.

There is further provided according to the invention use of a composition of the invention in the manufacture of a medicament for deodorising a wound.

A composition of the invention may be provided with instructions for use of the composition. For example, a composition of the invention may be packaged as a kit with the instructions.

Aspects of the invention are summarised in the following numbered clauses:

1. A composition comprising:

enzyme that is able to convert a substrate to release hydrogen peroxide; and substrate for the enzyme.

2. A composition according to clause 1, which does not comprise sufficient free water to allow the enzyme to convert the substrate.
3. A composition according to any preceding clause, which is in the form of a liquid or a gel.
4. A composition according to any preceding clause, which has a water activity of 0.7 or less, preferably 0.6 or less.
5. A composition according to any preceding clause, in which water is 20% or less, by weight, of the composition.
6. A composition according to any preceding clause, in which water is 15% or less, by weight, of the composition.
7. A composition according to any preceding clause, in which water is 10% or less, by weight, of the composition, preferably less than 10% by weight of the composition, more preferably 5% or less, by weight of the composition.
8. A composition according to any preceding clause in which water is at least 2% by weight of the composition.
9. A composition according to any preceding clause, in which water is at least 4%, by weight, of the composition.
10. A composition according to any preceding clause comprising solute.
11. A composition according to clause 10, wherein the solute consists of only one solute.
12. A composition according to clause 10, wherein the solute comprises at least two solutes.
13. A composition according to any of clauses 10 to 12, wherein the solute has a solubility of at least 100 g/100 g water at 20° C. and 1 atm
14. A composition according to any of clauses 10 to 12, wherein the solute has a solubility of at least 200 g/100 g water at 20° C. and 1 atm
15. A composition according to any of clauses 10 to 12, wherein the solute has a solubility of at least 300 g/100 g water at 20° C. and 1 atm.
16. A composition according to any of clauses 10 to 15, wherein the solute is, or comprises, a sugar or sugar derivative.
17. A composition according to clause 16, wherein the solute is, or comprises, a disaccharide or a monosaccharide.
18. A composition according to clause 17, wherein the solute is, or comprises, fructose.
19. A composition according to any of clauses 10 to 18, wherein the solute is distinct from the substrate.
20. A composition according to any of clauses 10 to 19, wherein the solute is at least 1%, by weight, of the composition.
21. A composition according to any of clauses 10 to 20, wherein the solute is at least 2%, by weight, of the composition.
22. A composition according to any of clauses 10 to 21, wherein the solute is at least 3% by weight, of the composition.
23. A composition according to any of clauses 10 to 22, wherein the solute is 10% or less, preferably 5% or less, by weight, of the composition.
24. A composition according to any of clauses 10 to 23, wherein the combined weight of the substrate and the solute is less than 20% by weight of the composition.
25. A composition according to any of clauses 10 to 24, wherein the combined weight of the substrate and the solute is 15% or less by weight of the composition.
26. A composition according to any of clauses 10 to 25, wherein the combined weight of the substrate and the solute is 10% or less by weight of the composition.
27. A composition according to any of clauses 10 to 26, wherein the combined weight of the substrate and the solute is at least 2% by weight of the composition.
28. A composition according to any of clauses 10 to 27, wherein the combined weight of the substrate and the solute is at least 5% by weight of the composition.
29. A composition according to any preceding clause, wherein the total weight of sugar in the composition is less than 20%.
30. A composition according to any preceding clause, wherein the total weight of sugar in the composition is 15% or less.
31. A composition according to any preceding clause, wherein the total weight of sugar in the composition is 10% or less, or less than 10%.
32. A composition according to any preceding clause, wherein the total weight of sugar in the composition is 7.5% or less.
33. A composition according to any preceding clause, wherein the total weight of sugar in the composition is 5% or less.

34. A composition according to any preceding clause, wherein the total weight of sugar or sugar derivative in the composition is 3% or less.

35. A composition according to any preceding clause, wherein the substrate consists of only one substrate.
36. A composition according to any preceding clause, wherein the substrate is less than 20% by weight of the composition.
37. A composition according to any preceding clause, wherein the substrate is 15% or less, by weight, of the composition.
38. A composition according to any preceding clause, wherein the substrate is 10% or less, by weight, of the composition,
39. A composition according to any preceding clause wherein the substrate is less than 10% by weight of the composition.
40. A composition according to any preceding clause, wherein the substrate is 7.5% or less, by weight of the composition.
41. A composition according to any preceding clause, wherein the substrate is at least 1%, by weight, of the composition.
42. A composition according to any preceding clause, wherein the substrate is at least 2%, by weight, of the composition.
43. A composition according to any preceding clause, wherein the substrate is at least 2.5% by weight, of the composition.
44. A composition according to any preceding clause, comprising non-aqueous solvent.
45. A composition according to clause 44, wherein the non-aqueous solvent is, or comprises, a polyol, optionally wherein the polyol is or comprises glycerol.
46. A composition according to clause 44 or clause 45, wherein the non-aqueous solvent is present in an amount of 20% to 60%, by weight, optionally 40% to 60% by weight, or 20% to 40%, by weight.
47. A composition according to any of clauses 44 or clause 45, wherein the non-aqueous solvent is present in an amount of at least 5%, by weight.
48. A composition according to any of clauses 44 to 45, wherein the non-aqueous solvent is present in an amount of at least 10%, by weight.
49. A composition according to any of clauses 44 to 45, wherein the non-aqueous solvent is present in an amount of at least 20%, at least 25%, greater than 50%, at least 60%, at least 70% or at least 80%, by weight.
50. A composition according to any of clauses 47 to 49, wherein the non-aqueous solvent is present in an amount of 60% or less, or 50% or less, by weight.
51. A composition according to any of clauses 47 to 50, wherein the non-aqueous solvent is present in an amount of 40% or less, by weight.
52. A composition according to any of clauses 47 to 51, wherein the non-aqueous solvent is present in an amount of 30% or less, by weight.
53. A composition according to any of clauses 47 to 52, wherein the non-aqueous solvent is present in an amount of 20% or less, by weight.
54. A composition according to any of clauses 47 to 53, wherein the non-aqueous solvent consists of only one non-aqueous solvent.
55. A composition according to any preceding clause, comprising antioxidant.
56. A composition according to clause 55, wherein the antioxidant consists of only one antioxidant.
57. A composition according to any of clauses 55 to 56, wherein the antioxidant is, or comprises, ascorbic acid, tocopherol or ascorbyl palmitate.
58. A composition according to clause 57, wherein the antioxidant is, or comprises, ascorbic acid.
59. A composition according to any of clauses 55 to 58, wherein the antioxidant is present in an amount of at least 0.01% by weight.
60. A composition according to any of clauses 55 to 59, wherein the antioxidant is present in an amount of at least 0.1% by weight.
61. A composition according to any of clauses 55 to 60, wherein the antioxidant is present in an amount of 1% by weight, or less.
62. A composition according to any of clauses 57 to 61, wherein the antioxidant is present in an amount of 0.5% by weight, or less.
63. A composition according to any preceding clause, comprising substantially no hydrogen peroxide, or comprising only trace amounts of hydrogen peroxide.
64. A composition according to any preceding clause, wherein the hydrogen peroxide is present at a concentration of 6 ppm or less.
65. A composition according to any preceding clause, wherein the hydrogen peroxide is present at a concentration of 3 ppm or less.
66. A composition according to any of clauses 1 to 63, wherein hydrogen peroxide is present at a concentration of 120 μM or less.
67. A composition according to clause 66 wherein hydrogen peroxide is present at a concentration of 100 μM or less.
68. A composition according to clause 67, wherein hydrogen peroxide is present at a concentration of 80 μM or less.
69. A composition according to any preceding clause, wherein the enzyme consists of only one enzyme.
70. A composition according to any preceding clause, wherein the enzyme is, or comprises, an oxidoreductase enzyme.
71. A composition according to any preceding clause, wherein the enzyme is, or comprises, glucose oxidase and the substrate is, or comprises, D-glucose.
72. A composition according to any preceding clause, wherein the enzyme is 0.01% to 0.5% by weight of the composition.
73. A composition according to any preceding clause, wherein the enzyme is 0.02% to 0.2% by weight of the composition.
74. A composition according to any preceding clause, wherein the enzyme is 25 to 2000 ppm of the composition, 25 to 2500 ppm of the composition, or 25 to 3000 ppm of the composition.

75. A composition according to any preceding clause, which is sterile.

76. A composition according to any preceding clause, which lacks catalase activity, or which comprises substantially no catalase activity.
77. A composition according to any preceding clause, which lacks peroxidase activity, or which comprises substantially no peroxidase activity.
78. A composition according to any preceding clause which does not comprise honey, or which comprises substantially no honey.
79. A composition according to any preceding clause, which does not comprise zinc oxide, or which comprises substantially no zinc oxide.
80. A composition according to any preceding clause, which is fluid.
81. A composition according to any preceding clause, which is a solution.
82. A composition according to any preceding clause which is a pharmaceutical grade composition.
83. A composition according to any preceding clause, wherein the enzyme is a purified enzyme.
84. A composition according to clause 83, wherein the enzyme has a mass purity of at least 95%.
85. A composition according to clause 84, wherein the enzyme has a mass purity of at least 98%.
86. A composition according to clause 85, wherein the enzyme has a mass purity of at least 99%.
87. A composition according to any preceding clause, wherein the substrate is a purified substrate.
88. A composition according to clause 87, wherein the substrate has a mass purity of at least 95%.
89. A composition according to clause 88, wherein the substrate has a mass purity of at least 98%.
90. A composition according to clause 89, wherein the substrate has a mass purity of at least 99%.
91. A composition according to clause 10, or any clause dependent on clause 10, wherein the solute is a purified solute.
92. A composition according to clause 91, wherein the solute has a mass purity of at least 95%.
93. A composition according to clause 92, wherein the solute has a mass purity of at least 98%.
94. A composition according to clause 93, wherein the solute has a mass purity of at least 99%.
95. A composition according to any preceding clause, which, at 1 hour, following a 1:1 dilution (by weight) with water, the level of hydrogen peroxide production increases by a factor of at least 5, at least 10, at least 20, at least 50, at least 100, at least 200, or at least 300.
96. A composition according to any preceding clause, which, at 24 hours, following a 1:1 dilution (by weight) with water, the level of hydrogen peroxide production increases by a factor of at least 5, at least 10, at least 20, at least 50, at least 100, at least 200, or at least 300.
97. A composition according to any preceding clause, comprising polymer, optionally wherein the polymer is a synthetic polymer.
98. A composition according to any of clause 97, wherein the polymer is, or comprises, polyethylene glycol (PEG).
99. A composition according to any of clauses 97 to 98, wherein the polymer comprises a first polymer and a second polymer, wherein the first polymer has a lower molecular weight than the second polymer, and optionally a third polymer of intermediate molecular weight to the first and second polymer.
100. A composition according to clause 100, wherein the composition comprising a first PEG polymer of molecular weight less than 1000, a second PEG polymer of molecular weight of 1000-3000, and a third PEG polymer of molecular weight greater than 3000.
101. A composition according to any of clauses 96 to 101, wherein: A) the polymer is added in an amount of at least 50% by weight, preferably at least 60% by weight; or B) the polymer is present in an amount of 30% to 70%, by weight.
102. A method for producing a composition, optionally wherein the composition is defined in any of clauses 1 to 101, comprising: contacting an enzyme that is able to convert a substrate to release hydrogen peroxide with a substrate for the enzyme.
103. A method according to clause 102, wherein the composition has a water activity of 0.7 or less, preferably 0.6 or less; or wherein the method comprises adding water such that the water activity is 0.7 or less, preferably 0.6 or less.
104. A method according to any of clauses 102 to 103, wherein water is 20% or less, by weight, of the composition; or wherein the method comprises adding water such the water is 20% or less, by weight, of the composition.
105. A method according to any of clauses 102 to 104, wherein water is 15% or less, by weight, of the composition; or wherein the method comprises adding water such that the water is 15% or less, by weight, of the composition.
106. A method according to any of clauses 102 to 105, wherein water is less than 10%, by weight, of the composition; or wherein the method comprises adding water such that the water is less than 10%, by weight, of the composition, preferably wherein water is 5% or less, by weight, of the composition; or wherein the method comprises adding water such that the water is 5% or less by weight, of the composition.
107. A method according to any of clauses 102 to 106, wherein the water is at least 2%, by weight, of the composition; or wherein the method comprises adding water such that the water is at least 2%, by weight, of the composition.
108. A method according to any of clauses 102 to 107, comprising adding solute.
109. A method according to clause 108 wherein the solute is in the form of a sugar.
110. A method according to clause 109, wherein the solute is, or comprises, a disaccharide or a monosaccharide
111. A method according to clause 110, wherein the solute is, or comprises, fructose.
112. A method according to any of clauses clause 108 to 111, wherein the solute has a solubility of at least 100 g/100 g water at 20° C. and 1 atm, preferably at least 200 g/100 g water at 20° C. and 1 atm, more preferably at least 300 g/100 g water.
113. A method according to any of clauses 108 to 112, wherein the solute is added such that it is at least 1%, by weight, of the composition.
114. A method according to any of clauses 108 to 113, wherein the solute is added such that it is at least 2%, by weight, of the composition.

115. A method according to any of clauses 108 to 114, wherein the solute is added such that it is at least 3%, by weight, of the composition.

116. A method according to any of clauses 108 to 115, wherein the solute is added such that it is 10% or less, by weight, of the composition, preferably 5% or less by weight of the composition.
117. A method according to any of clauses 108 to 116, wherein solute and substrate are added such that the combined weight of the substrate and the solute is less than 20% by weight of the composition.
118. A method according to any of clauses 108 to 117, wherein the solute and substrate are added such that combined weight of the substrate and the solute is 15% or less by weight of the composition.
119. A method according to any of clauses 108 to 118, wherein the solute and substrate are added such that combined weight of the substrate and the solute is 10% or less by weight of the composition.
120. A method according to any of clauses 108 to 119, wherein the solute and substrate are added such that combined weight of the substrate and the solute is at least 2% by weight of the composition, preferably at least 5% by weight of the composition.
121. A method according to any of clauses 102 to 120, wherein sugar is added such that the total amount of sugar in the composition is less than 20% by weight.
122. A method according to any of clauses 102 to 121, wherein sugar or sugar derivative is added such that the total amount of sugar in the composition is 15% or less by weight.
123. A method according to any of clauses 102 to 122, wherein sugar or sugar derivative is added such that the total amount of sugar or sugar derivative in the composition is at least 10% or less by weight.
124. A method according to any of clauses 102 to 123, wherein the substrate is added such that it is less than 20%, by weight, of the composition.
125. A method according to any of clauses 102 to 124, wherein the substrate is added such that it is 15% or less, by weight, of the composition.
126. A method according to any of clauses 102 to 125, wherein the substrate is added such that it is 10% or less, by weight, of the composition.
127. A method according to any of clauses 102 to 126, wherein the substrate is added such that it is less than 10% by weight of the composition.
128. A method according to any of clauses 102 to 127, wherein the substrate is added such that it is 7.5% or less, by weight, of the composition.
129. A method according to any of clauses 102 to 128, wherein the substrate is added such that it is at least 1%, by weight, of the composition.
130. A method according to any of clauses 102 to 129, wherein the substrate is added such that it is at least 2% by weight, of the composition.
131. A method according to any of clauses 102 to 130 in which the enzyme is a purified enzyme.
132. A method according to clause 131, wherein the enzyme has a mass purity of at least 95%, preferably at least 98%.
133. A method according to any of clauses 102 to 132 wherein the substrate is a purified substrate.
134. A method according to clause 133, wherein the substrate has a mass purity of least 95%, preferably at least 98%.
135. A method according to clause 111, or any clause dependent on clause 111, wherein the solute is a purified solute.
136. A method according to clause 135, wherein the solute has a mass purity of at least 95%, preferably at least 98%.
137. A method according to any of clauses 102 to 136, comprising adding non-aqueous solvent.
138. A method according to clause 137, wherein the non-aqueous solvent is, or comprises, a polyol.
139. A method according to clause 138, wherein the polyol is, or comprises, glycerol.
140. A method according to any of clauses 137 to 139, wherein the non-aqueous solvent is added in an amount of at least 5%, by weight.
141. A method according to any of clauses 137 to 140, wherein the non-aqueous solvent is added in an amount of at least 10%, by weight.
142. A method according to any of clauses 137 to 141, wherein the non-aqueous solvent is added in an amount of at least 20%, at least 25%, greater than 50%, at least 60%, at least 70% or at least 80%, by weight.
143. A method according to any of clauses 137 to 142, wherein the non-aqueous solvent is added in an amount of 60 or less, or 50% or less, by weight.
144. A method according to any of clauses 137 to 143, wherein the non-aqueous solvent is added in an amount of 40% or less, by weight.
145. A method according to any of clauses 137 to 144, wherein the non-aqueous solvent is added in an amount of 30% or less, by weight.
146. A method according to any of clauses 137 to 145, wherein the non-aqueous solvent is added in an amount of 20% or less, by weight.
147. A method according to any of clauses 137 to 146, wherein the non-aqueous solvent consists of only one non-aqueous solvent.
148. A method according to any of clauses 102 to 147, comprising adding antioxidant.
149. A method according to clause 148, wherein the antioxidant is ascorbic acid, tocopherol or ascorbyl palmitate.
150. A method according to clause 148 or 149, wherein the antioxidant consists of only one antioxidant.
151. A method according to any of clauses 148 to 150, wherein the antioxidant is added in an amount of at least 0.01% by weight.
152. A method according to any of clauses 148 to 151, wherein the antioxidant is added in an amount of at least 0.1% by weight.
153. A method according to any of clauses 148 to 152, wherein the antioxidant is added in an amount of 2% by weight, or less.
154. A method according to any of clauses 148 to 153, wherein the antioxidant is added in an amount of 1% by weight, or less.
155. A method according to any of clauses 102 to 154, comprising adding polymer.
156. A method according to any of clauses 102 to 155, wherein the polymer is a synthetic polymer.
157. A method according to clause 156, wherein the polymer is, or comprises polyethylene glycol (PEG).
158. A method according to any of clauses 102 to 157, wherein the polymer comprises a first polymer and a second polymer, wherein the first polymer has a lower molecular weight than the second polymer.
159. A method according to clause 158, wherein the polymer comprises a third polymer of intermediate molecular weight to the first and second polymer.
160. A method according to clause 159, wherein the polymer a first PEG polymer of molecular weight less than 1000, a second PEG polymer of molecular weight of 1000-3000, and a third PEG polymer of molecular weight greater than 3000.
161. A method according to any of clauses 155 to 160, wherein the polymer is added in an amount of at least 50% by weight, or at least 60% by weight.
162. A method according to any of clauses 155 to 160, wherein the polymer is added in an amount of 30% to 70%, by weight.
163. A method according to any of clauses 102 to 162, wherein the composition is formulated such that there is substantially no hydrogen peroxide in the composition.
164. A method according to any of clauses 102 to 263, wherein the composition is formulated such that hydrogen peroxide is present in the composition at a concentration of 6 ppm or less, 3 ppm or less, 2 ppm or less, or 1 ppm or less.
165. A method according to any of clauses 102 to 164, wherein the composition is formulated such that hydrogen peroxide is present in the composition at a concentration of 120 μM or less, preferably 100 μM or less, more preferably 80 μM or less.
166. A method according to any of clauses 102 to 165, comprising adding buffer.
167. A method according to any of clauses 102 to 166, wherein the composition is formulated such that it is at a pH of 6.5 to 7.5.
168. A method according to any of clauses 102 to 167, wherein the enzyme is an oxidoreductase enzyme.
169. A method according to clause 168, wherein the enzyme is glucose oxidase and the substrate is D-glucose.
170. A method according to any of clauses 102 to 169, wherein the composition is formulated such the enzyme is 0.001% to 0.5%, by weight, of the composition.
171. A method according to clause 170, wherein the composition is formulated such that the enzyme is 0.0025 to 0.2% enzyme, by weight, of the composition.
172. A method according to any of clauses 102 to 171, wherein the composition is formulated such that the enzyme is 25 to 2500 ppm of the composition.
173. A method according to any of clauses 102 to 172, wherein the composition is sterile, or wherein the method comprises sterilising to form a sterile composition, optionally by exposing applying gamma irradiation or electron beam irradiation.
174. A method according to any of clauses 102 to 173, wherein the composition is formulated such that it lacks catalase activity.
175. A method according to any of clauses 102 to 174, wherein the composition is formulated such that is lacks peroxidase activity.
176. A method according to any of clauses 102 to 175, wherein the composition is formulated such that it does not comprise honey.
177. A method according to any of clauses 102 to 176, wherein the composition is formulated such that it does not comprise zinc oxide.
178. A composition according to any of clauses 1 to 101, for use as a medicament.
179. A composition according to any of clauses 1 to 101, for use in prevention, treatment or amelioration of a microbial infection.
180. A composition according to any of clauses 1 to 101, for use in treatment of a wound.
181. A composition according to clause 180, wherein the wound is a chronic wound.
182. A wound dressing which comprises a dressing material for dressing a wound, and a composition according to any preceding clause.
183. A package or container comprising a composition as defined in any of clauses 1 to 96.
184. A package or container according to clause 98, which is air-tight and/or opaque.
185. A dispensing device comprising a composition as defined in any of clauses 1 to 96.
186. A device according to clause 100, wherein the device is a spraying device, a tube or a syringe.

Preferred embodiments of the invention are now described, by way of example only, with reference to the accompanying drawings in which:

FIG. 1 shows a hydrogen peroxide production profile, following dilution, of a formulation comprising 31% glucose, 52% fructose, 16.9% water and 0.1% glucose oxidase;

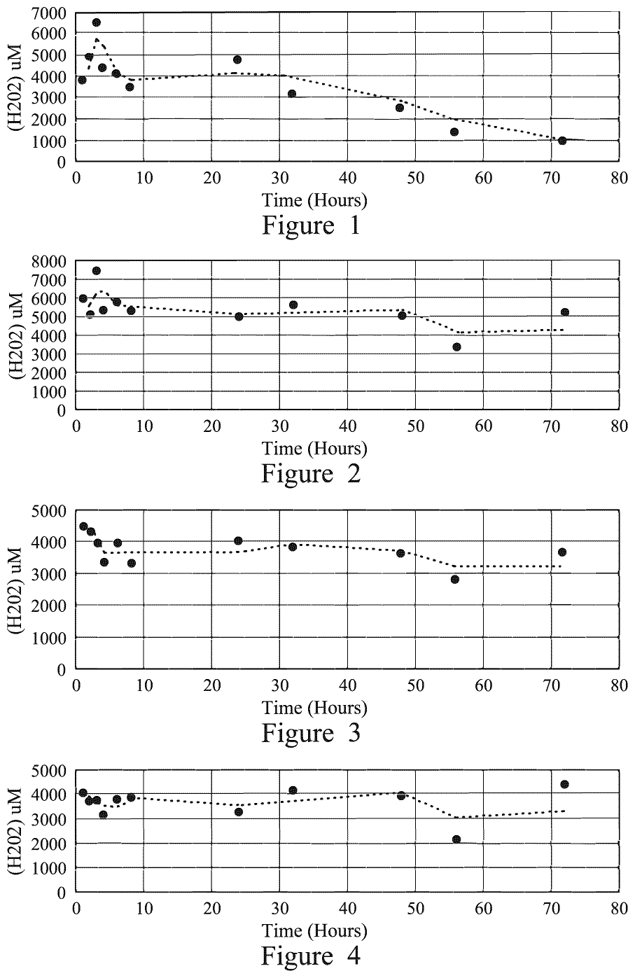

FIG. 2 shows a hydrogen peroxide production profile, following dilution, of a formulation comprising 31% glucose, 52% fructose, 9.9% water, 7% glycerol and 0.1% glucose oxidase;

FIG. 3 shows a hydrogen peroxide production profile, following dilution, of a formulation comprising 5% glucose, 78% fructose, 16.9% water and 0.1% glucose oxidase;

FIG. 4 shows a hydrogen peroxide production profile, following dilution, of a formulation comprising 5% glucose, 83% fructose, 11.9% water and 0.1% glucose oxidase;

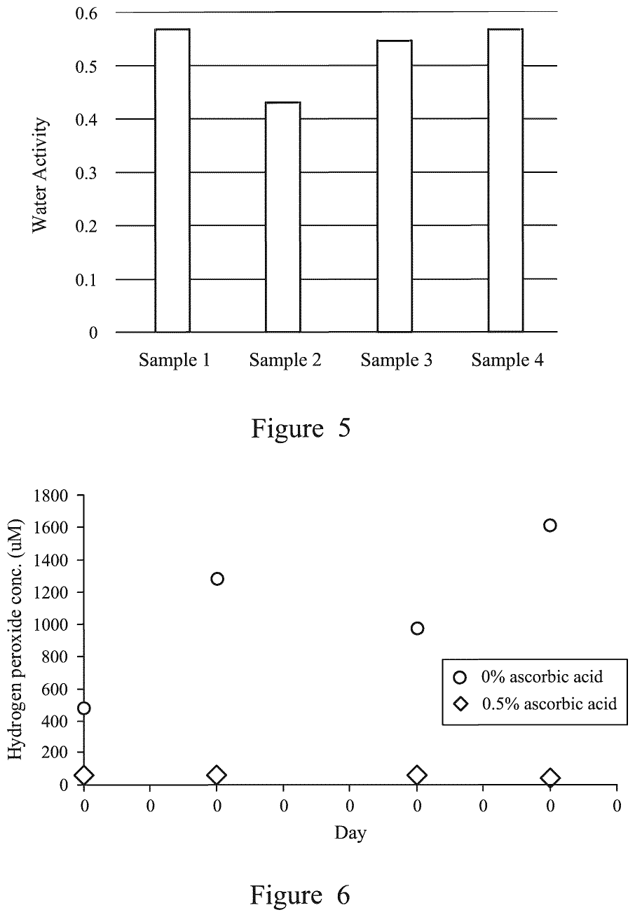

FIG. 5 shows the water activities of the formulations of FIGS. 1 to 4;

FIG. 6 shows levels of hydrogen peroxide in formulations comprising 5% glucose, 78% fructose, 9.9% water, 7% glycerol and 0.025% glucose oxidase, both with and without 0.5% ascorbic acid, over a period of 7 days;

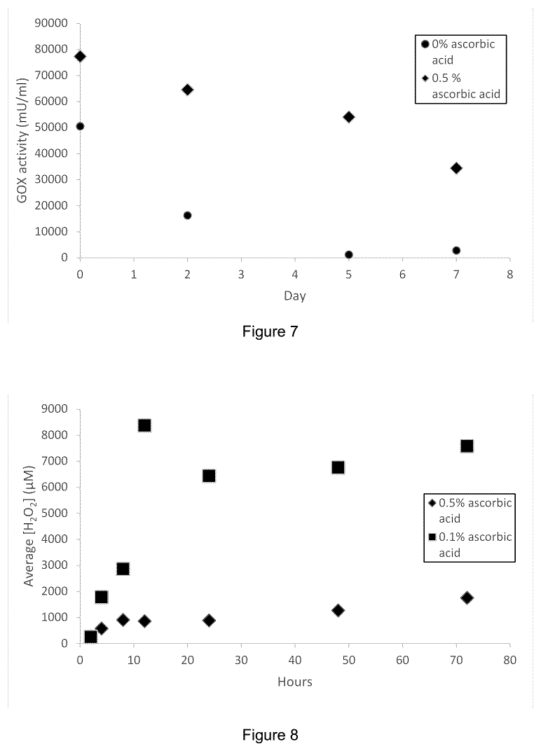

FIG. 7 shows glucose oxidase activity levels of the formulations of FIG. 5, over a period of 7 days;

FIG. 8 shows a hydrogen peroxide production profile, following dilution, of a formulation comprising 5% glucose, 78% fructose, 9.9% water, 7% glycerol and 0.025% glucose oxidase, with both 0.1% ascorbic acid and 0.5% ascorbic acid;

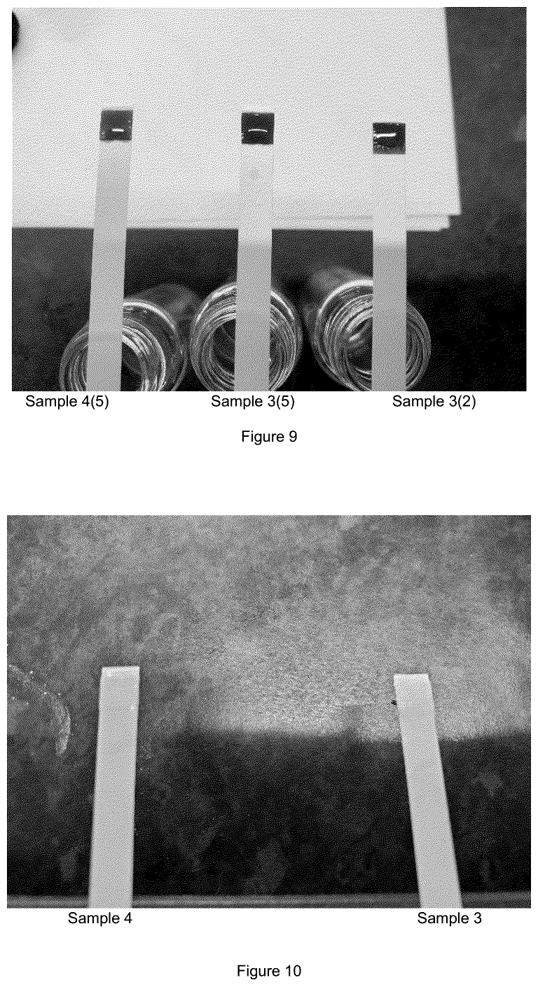

FIG. 9 shows the results of a hydrogen peroxide ‘blue stick’ test following dilution of two formulations each comprising glucose, glucose oxidase, polyethylene glycol and glycerine.

FIG. 10 shows the results of a hydrogen peroxide ‘blue stick test’ of the formulations of FIG. 9, but without dilution, i.e. at ‘rest’.

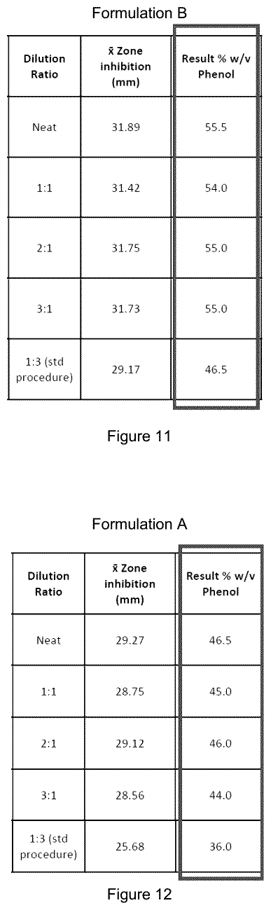

FIG. 11 shows the results of microbiological well diffusion assays for a formulation comprising glucose, glucose oxidase, polyethylene glycol and glycerine;

FIG. 12 shows the results of microbiological well diffusion assays for another formulation comprising glucose, glucose oxidase, polyethylene glycol and glycerine;

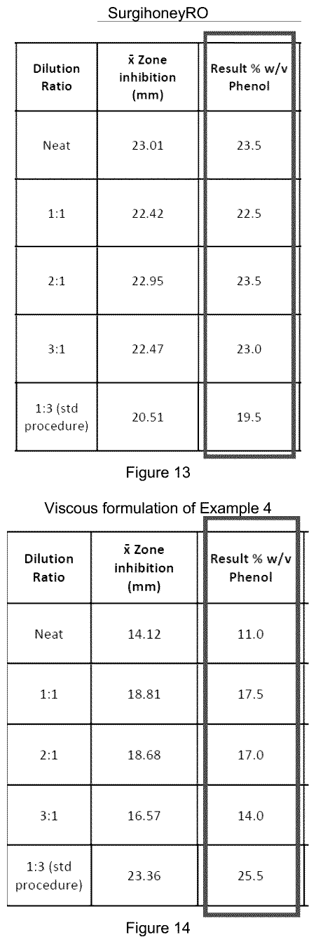

FIG. 13 shows the results of microbiological well diffusion assays for Surgihoney; and

FIG. 14 shows the results of microbiological well diffusion assays for another formulation comprising glucose, glucose oxidase, polyethylene glycol and glycerine.

### EXAMPLE 1

### Methods

Samples were formulated as follows (all amounts are expressed as % by weight).

TABLE 1

| | | | | Glyc- | Glucose |
|---|---|---|---|---|---|
| | Glucose | Fructose | Water | erol | Oxidase |
| Sample/formulation 1 | 31 | 52 | 16.9 | 0 | 0.1 |
| Sample/formulation 2 | 31 | 52 | 9.9 | 7 | 0.1 |
| Sample/formulation 3 | 5 | 78 | 16.9 | 0 | 0.1 |
| Sample/formulation 4 | 5 | 83 | 11.9 | 0 | 0.1 |

### Stability Study

On the first day of the study the samples were placed at 25° C. in the incubator. Each day of the study (day 1, 3, and 6) samples were taken from the incubator, mixed by stirring and then analysed for glucose oxidase activity (TM-601) and hydrogen peroxide levels (TM-781). All dilutions were performed gravimetrically to ensure the highest level of accuracy. In addition on day 1 and 6 each of the samples was activated by the addition of an equal mass of water and incubated at 37° C. for up to 72 hours. At specified times the level of hydrogen peroxide was determined by testing an aliquot of the incubated activated solution.

### Glucose Oxidase Assay (TM-601)

Approximately 1 g of samples were weighed into a 15 ml tube and diluted with 9 mL of phosphate buffered saline (PBS). All weights were measured using a 5 place balance for calculation of actual dilutions. The samples were vortex mixed to ensure that the entire sample was solubilised and then diluted serially to give a dilution between 5,000 and 100,000 times for analysis. 50 1-JL of sample, standard curve point, blank or quality control was pipetted into a 96 well plate and the reaction started by addition of 50 1-JL of substrate. The change in absorbance at 570 nm was monitored over a half an hour time period and the rate of change of OD used to calculate the glucose oxidase activity by comparison with the standard curve.

### Hydrogen Peroxide Assay (TM-781)

Approximately 0.3 g of samples were weighed into 1.5 ml microfuge tubes and diluted with and equal mass of 99% ethanol. The solutions were physically mixed using a sterile pipette tip prior to being centrifuged to remove precipitated material. The ethanol supernatant was diluted through a series of three dilutions (500 to 10,000 times) with PBS. 50 1-JL of sample, standard curve point or blank was pipetted into a 96 well plate and the reaction started by addition of 50 1-JL of substrate. After five minutes the absorbance at 570 nm was determined and used to produce the standard curve and quantify the level of hydrogen peroxide in each sample.

### Activated Hydrogen Peroxide Level Determination

In order to determine the level of hydrogen peroxide when the formulation was activated by contact with water approximately 0.65 g of sample was mixed well with an equal mass of water and made homogeneous by vortex mixing. This solution was incubated at 37° C. for up to 72 hours. Periodically samples were taken and extracted with 99% ethanol to precipitate the protein and sugar. Then following a dilution with PBS the samples were analyzed for hydrogen peroxide level using the Amplex Red hydrogen peroxide assay (TM-781) as detailed above.

### Results

FIGS. 1, 2, 3 and 4 show graphical representations of the change in hydrogen peroxide concentration with time following activation of samples 1, 2, 3 and 4, respectively, by addition of water.

It can be observed how a significant reduction in the level of glucose can still result in a composition that is able to produce hydrogen peroxide over an extended period of time following dilution.

It is noted that the water activity of all samples was less than 0.6 (See FIG. 5).

It is also noted that Sample 4 was visually biphasic with significant amounts of precipitated sugar.

From the stability study, the following changes in glucose oxidase activity were observed.

TABLE 2

| | % change in glucose | % change in glucose |
|---|---|---|
| | oxidase activity | oxidase activity |
| | (day 1 to day 3) | (day 1 to day 6) |
| Sample/Formulation 1 | −9.55 | −22.05 |
| Sample/Formulation 2 | −2.22 | +1.78 |

These data indicates that inclusion of a non-aqueous solvent such as glycerol can reduce loss of glucose oxidase activity.

### EXAMPLE 2

TABLE 3

| | | | | | Glucose |
|---|---|---|---|---|---|
| | Glucose | Fructose | Water | Glycerol | Oxidase |
| Sample 1 | 31 | 52 | 9.9 | 7 | 0.1 |
| (Variant 1) | | | | | |
| Sample 2 | 5 | 78 | 9.9 | 7 | 0.1 |
| (Variant 2) | | | | | |

All amounts % by weight

### Methods

In order to prepare a range of products containing different levels of hydrogen peroxide, the samples were mixed according to the tables below, with the masses being recorded to calculate the exact dilution being performed

### Sample 1:

TABLE 4

| | Target | Mass of | | Mass of | Dilution | Actual |
|---|---|---|---|---|---|---|
| Dilution | [Gox] | Sample | | variant | Factor | [GoX] |
| number | (%) | (g) | Description of sample | (g) | (times) | (%) |
| 1 | 0.05 | 20.59269 | 0.1% GoX variant_{—} | 20.59868 | 2.0 | 0.0500 |
| 2 | 0.025 | 20.24319 | 0.05% (dilution1) | 20.30243 | 2.0 | 0.0250 |
| 3 | 0.005 | 10.01538 | 0.025% (dilution2) | 40.05496 | 5.0 | 0.0050 |
| 4 | 0.003 | 30.01160 | 0.005% (dilution 3) | 22.10454 | 1.7 | 0.0029 |
| 5 | 0.001 | 10.24168 | 0.003% (dilution4) | 20.12446 | 3.0 | 0.0010 |

### Sample 2:

TABLE 5

| | Target | Mass of | | Mass of | Dilution | Actual |
|---|---|---|---|---|---|---|
| Dilution | [GoX] | Sample | | variant | Factor | [GoX] |
| number | (%) | (g) | Description of sample | (g) | (times) | (%) |
| 6 | 0.05 | 20.90064 | 0.1% GoX variant | 20.72883 | 2.0 | 0.0502 |
| 7 | 0.025 | 20.11057 | 0.05% (dilution 6) | 20.33254 | 2.0 | 0.0250 |
| 8 | 0.005 | 10.39779 | 0.025% (dilution 7) | 40.43689 | 4.9 | 0.0051 |
| 9 | 0.003 | 30.29630 | 0.005% (dilution 8) | 20.02033 | 1.7 | 0.0031 |
| 10 | 0.001 | 10.35204 | 0.003% (dilution 9) | 20.06589 | 2.9 | 0.0010 |

Each of these samples was tested for the level of resting hydrogen peroxide using the Amplex Red hydrogen peroxide assay (TM-781, see Example 1), as well as set up for activation by addition of an equal mass of water and incubation at 37° C. for up to 24 hours.

The level of activated hydrogen peroxide was determined as described in Example 1.

TABLE 6

| | Resting | 1 hour | |
|---|---|---|---|
| | [H₂O₂] μM | [H₂O₂] μM | Ratio |
| Sample 1 0.025% GOX | 65.7 | 2425.38 | 369 |
| Sample 1 0.005% GOX | 21.9 | 11170.69 | 510 |
| Sample 1 0.003% GOX | 17.1 | 9506.756 | 557 |
| Sample 1 0.001% GOX | 10.6 | 4582.596 | 432 |
| Sample 2 0.025% GOX | 43.2 | 17097.48 | 395 |
| Sample 2 0.005% GOX | 26.9 | 9800.408 | 364 |
| Sample 2 0.003% GOX | 27.4 | 8444.577 | 308 |
| Sample 2 0.001% GOX | 27.8 | 2842.829 | 102 |

### EXAMPLE 3

TABLE 7

| | | | | Glyc- | Glucose | Ascorbic |
|---|---|---|---|---|---|---|
| | Glucose | Fructose | Water | erol | Oxidase | Acid |
| Sample 1 | 5 | 78 | 9.9 | 7 | 0.1 | 0.5 |
| (Variant 1) | | | | | | |
| Sample 2 | 5 | 78 | 9.9 | 7 | 0.1 | 0.1 |
| (Variant 2) | | | | | | |

All amounts % by weight

In order to prepare samples containing different levels of glucose oxidase, the samples were prepared by gravimetric mixing of a 0.1% and 0% glucose oxidase base preparations, with the masses being recorded to calculate exact dilutions used.

Each of the samples plus the supplied 0.1% GoX for each were then tested for the level of resting hydrogen peroxide and glucose oxidase activity using the Amplex Red hydrogen peroxide and glucose oxidase assays (TM-781 and TM-601; see above) respectively.

### Resting and Stability Hydrogen Peroxide and Glucose Oxidase Levels

For glucose oxidase determination, approximately 0.1 g of each sample was weighed into 1.5 mL microfuge tubes and diluted with 900 μPBS. For hydrogen peroxide, approximately 0.3 g of each sample was weighed into 1.5 ml microcentrifuge tubes and diluted with an equal mass of 99% ethanol. For both, the solutions were physically mixed using a sterile pipette tip prior to being centrifuged to remove precipitated material and then diluted to a suitable level using PBS.

50 μl of sample, standard curve point (hydrogen peroxide or glucose oxidase as required) or blank was pipetted into a 96 well plate and the reaction started by addition of 50 μl of the relevant substrate. For both assays, the absorbance at 570 nm was determined, for glucose oxidase, every 2 minutes for a total of 30 minutes and after 5 minutes for hydrogen peroxide.

This was then used to produce the standard curve for each assay and quantify both the glucose oxidase activity and hydrogen peroxide level in each sample. The samples were also stored at 25° C. for 7 days and tested at 2, 5 and 7 days thereafter.

### 7 Day Stability Study of 0.25% Glucose Oxidase Formulation Containing Different Levels of Ascorbic Acid

TABLE 8

Time (days)

| Ascorbic acid % | 0 | 2 | 5 | 7 |
|---|---|---|---|---|
| 0% ascorbic acid | 476.6 | 1278.8 | 981.3 | 1618.8 |
| 0.5% ascorbic acid | 50.1 | 61.6 | 59.7 | 69.1 |

[H₂O₂] μM

The results are illustrated in FIG. 6. The resting levels of hydrogen peroxide were reduced by the presence of ascorbic acid.

TABLE 9

Time (days)

| Ascorbic acid % | 0 | 2 | 5 | 7 |
|---|---|---|---|---|
| 0% ascorbic acid | 50474.7 | 16252.9 | 1190.1 | 2756.7 |
| 0.5% ascorbic acid | 77285.3 | 64534.6 | 54056.1 | 34364.4 |

GOX activity (mU/ml)

The results are illustrated in FIG. 7. In the presence of ascorbic acid, the glucose oxidase has a higher initial activity and glucose oxidase activity is maintained at a higher level over time, over the testing period.

### Simulated Wound Hydrogen Peroxide Level Determination

A simulated wound was established by weighing 0.5g of the sample into a microfuge tube and centrifuging briefly to collect the gel at the bottom of the tube. On top of this was added 0.5 ml of horse serum and the tube incubated at 30° C. for up to 72 hours. During this time, 10 μl samples of the liquid supernatant (after mixing by inversion) were removed into either 140 μl of 99% ethanol for hydrogen peroxide measurement.

The hydrogen peroxide assay (TM-781) vortex mixed the sample briefly, followed by centrifugation at 12,000 rpm for 2 minutes to pellet precipitated material. The ethanol supernatant was further diluted with PBS and tested for hydrogen peroxide levels the Amplex Red assay kit.

### 72 Hour Hydrogen Peroxide Profiles

TABLE 10

| | 0.1% ascorbic acid | | 0.5% ascorbic acid | |
|---|---|---|---|---|
| Time | Average [H202] | Standard | Average [H202] | Standard |
| (hours) | (μM) | deviation | (μM) | deviation |
| 2 | 247 | 105.5 | 268.1 | 140 |
| 4 | 1777.8 | 801.3 | 573.6 | 16.1 |
| 8 | 2856.7 | 801.3 | 902.1 | 22.1 |
| 12 | 8364.2 | 457 | 851.2 | 127.3 |
| 24 | 6431 | 524.8 | 877.5 | 199.6 |
| 48 | 6754.2 | 326 | 1263.3 | 135.7 |
| 72 | 7568.8 | 46.4 | 1749.8 | 320.7 |

0.025% Glucose oxidase

The results are illustrated in FIG. 8.

### EXAMPLE 4—Viscous Liquid

### Formula

| Ingredient | % (by weight) |
|---|---|
| PEG 4000 | 4 |
| PEG 1500 | 15 |
| PEG 300 | 46.595 |
| Glycerin | 24 |
| Fructose | 3 |
| Ascorbic Acid | 0.25 |
| Glucose | 3 |
| Glucose oxidase | 0.005 |
| TRIS Buffer | 4.15 |
| | 100 |

| TRIS Buffer | Qty |
|---|---|
| TRIS | 1.211 g |
| Water | to 100 ml |
| HCl | to pH 7.0 |

### Process

Dissolve PEG polymers together with mixing
Dissolve glucose oxidase in 2g water
Add glycerin, glucose and fructose
Add glucose oxidase solution and remaining buffer

### EXAMPLE 5—Liquid Spray

### Formula

| Ingredient | % |
|---|---|
| PEG 300 | 66.9 |
| Glycerin | 15 |
| Glucose | 3 |
| GoX Powder | 0.1 |
| TRIS Buffer | 15 |
| | 100 |

| TRIS Buffer | Qty |
|---|---|
| TRIS | 1.2114 g |
| Water | to 100 ml |
| HCl | to pH 7.0 |

### Process

Weigh 35 g water
Using the small flea stirrer dissolve glucose (warm if needed)
Weigh PEG into a beaker
Add with stirring the glucose solution
Add glycerin
Weigh 10 g water add the GoX to 10 g water and stir until dissolved
Add Gox Solution to main liquid
Mix well for 15 mins
Pack 20ml into bottles and label

### EXAMPLE 6

A composition was formulated as indicated in Example 4, except that the glucose oxidase was added in dry, powder form at 0.025% by weight, and the TRIS buffer was added at 4% by weight.

### Hydrogen Peroxide Assay

The level of hydrogen peroxide both prior to dilution and following dilution was assessed according to the method in Example 1, above (TM-781).

The results are shown in Table 11, below.

TABLE 11

| | Hydrogen peroxide concentration |
|---|---|
| Time of measurement | (μM) |
| Resting (i.e. before dilution) | 28.9 |
| 1 hour following dilution | 2487.2 |
| 24 hours following dilution | 2456.0 |
| 48 hours following dilution | 3883.6 |
| 96 hours following dilution | 5480.2 |

### Water Activity

Water activity was measured at 0.279 at 25° C.

### Glucose Oxidase Activity

Glucose oxidase activity was measured according to the method in Example 1 (TM-601).

The results are shown in Table 12, below.

TABLE 12

| Time of measurement | Glucose oxidase activity (mU/mL) |
|---|---|
| Resting (i.e. before dilution) | 51356.3 |
| 92 hours following dilution | 27515.4 |

### EXAMPLE 7

A composition was formulated as follows.

| Formulation | % wt/wt |
|---|---|
| Glycerol | 84.00 |
| Glucose | 3.00 |
| Glucose Oxidase | 0.10 |
| Monosodium phosphate | qs |
| Disodium phosphate | qs |
| Water | 12.9 |

The glycerol, glucose, monosodium phosphate and disodium phosphate were all pharmaceutical grade according to the European Pharmacopoeia (EP).

This composition was found to be less bitter tasting than the liquid spray composition of Example 5.

### EXAMPLE 8

The formulations of Examples 5 and 7 were trialled privately in the treatment and prevention of COVID-19.

In one household of five members, one member contracted COVID-19. All five members initiated self-isolation for 14 days Each member of the family administered the spray formulation as follows: one squirt to each nostril and three squirts to the mouth/throat, three times per day

After 14 days, all family members tested negative for the virus and the spray had been well tolerated. There was no increase in viral load in the positive case and no transmission to the other members of the family.

### EXAMPLE 9

### Formulation A (Sample 4)

| Ingredient | % (weight) |
|---|---|
| PEG 1500 | 15 |
| PEG 600 | 25 |
| Glycerin | 53 |
| Glucose | 3 |
| Glucose oxidase | 0.005 |
| Water | 4 |

### Formulation B (Sample 3)

| Ingredient | % (weight) |
|---|---|
| PEG 1500 | 15 |
| PEG 600 | 46.5 |
| Glycerin | 31.5 |
| Glucose | 3 |
| Glucose oxidase | 0.005 |
| Water | 4 |

### Methodology

Formulations A and B were prepared on the same day and then allowed to stand overnight before testing. At rest, testing involved insertion of blue stick into the RO-101 directly, activation involved a dilution of the formulations with water, either 1 g, made to total 5 g with water or 1 g gel made to 2 g with water as shown in the results table below.

### Results

TABLE 13

| | Blue Stick test (ppm HP) | | | | | | | | |
|---|---|---|---|---|---|---|---|---|---|
| | | Activation | | | | | | | |
| Sample ref | Rest | Time 0 | 10 min | 30 min | 1 hr | 3 hr | 24 hr | 48 Hr | 72 Hr |
| Sample 4 | 0 | 3 | 10 | 10 | 30 | 30 | 100 | 100 | 100 |
| (diluted | | | | | | | | | |
| 1 g->5 g) | | | | | | | | | |
| Sample 3(5) | 0 | 10 | 10 | 10 | 30 | 30 | 100 | 100 | 100 |
| (diluted | | | | | | | | | |
| 1 g->5 g) | | | | | | | | | |
| Sample 3(2) | 0 | 10 | 10 | 30 | 100 | 100 | 100 | 100 | 100 |
| (diluted | | | | | | | | | |
| 1 g->2 g) | | | | | | | | | |

Sample 3(5) gave a generally stronger blue stick response than equivalent sample 4, but in turn the hydrogen peroxide from 3(2) was significantly stronger then 3(5) as shown in FIG. 9.

The change in colour for all samples was instant, though darkening of the stick continues over a few minutes. Sample 3 (2) had a different ‘shade’ of colour to its equivalent 3 (5), which is described as a ‘dirty blue’.

The samples at rest were tested and there was no development of blue colour on the stick after minutes. This is shown in FIG. 10. A very gradual browning of the test sticks was noted.

### EXAMPLE 10

Microbiological well-diffusion assays were carried out for Formulations A and B described in Example 9.

Formulations were diluted with de-ionised water, where appropriate, and transferred to wells on an agar plate that has been inoculated with a strain of Staphylococcus aureus. The plates were then incubated at 37° C. for 16 -18 hours after which the zones of inhibition were measured using digital callipers. These zones were compared with solutions of phenol of known concentration that are inoculated on to the same plate.

The mean diameter of all of the readings taken from the zone of inhibition for each standard, control and sample solution. The mean diameter is squared and a linear x y scatter graph is constructed by plotting the squared mean diameter for each standard solution on the y axis and the standard concentration (in w/v Phenol) on the x axis.

The Slope and the Intercept of the graph is calculated.

The % w/v Phenol of each sample is calculated using the following calculation:

$$TA ( \% w / v Phenol ) = \frac{{[ Mean diameter ]}^{2} - Intercept}{Slope} \times 4.69$$

The results are shown in FIGS. 11 and 12.

Results from equivalent tests carried out with SurgihoneyRO and the viscous liquid of example 4 are shown in FIGS. 13 and 14, respectively.

1. A liquid or gel composition, comprising:
enzyme that is able to convert a substrate to release hydrogen peroxide; and
substrate for the enzyme,
wherein the composition does not comprise sufficient free water to allow the enzyme to convert the substrate, or has a water activity (a_{w}) of 0.7 or less, and
wherein:
A) the composition comprises polymer, and i) there is greater than 50%, by weight, of the polymer in the composition; and/or ii) there is less than 20%, by weight, of sugar in the composition; or
B) the composition comprises non-aqueous solvent and does not comprise honey, wherein there is 5% or less, by weight, of the substrate in the composition, and wherein i) there is greater than 50%, by weight, of the non-aqueous solvent in the composition and/or ii) there is less than 20%, by weight, of sugar in the composition.

2. A composition according to claim 1, wherein there is greater than 50%, by weight, of the polymer in the composition.

3. A composition according to any preceding claim, wherein there is less than 20%, by weight, of sugar in the composition.

4. A composition according to any preceding claim, wherein there is 10% or less, by weight, of sugar in the composition.

5. A composition according to any preceding claim, wherein there is 6% or less, by weight, of sugar in the composition.

6. A composition according to any preceding claim, wherein there is less than 10% by weight of water in the composition, preferably 5% or less by weight of water in the composition.

7. A composition according to any preceding claim, which has a water activity of 0.6 or less, preferably 0.5 or less.

8. A composition according to any preceding claim, wherein there is 10% or less, by weight, of the substrate in the composition.

9. A composition according to any preceding claim, wherein there is 5% or less, by weight, of the substrate in the composition.

10. A composition according to any preceding claim, wherein there is at least 1%, by weight, of the substrate in the composition.

11. A composition according to any preceding claim, wherein the substrate is, or comprises, glucose and the enzyme is, or comprises, glucose oxidase.

12. A composition according to any preceding claim, comprising solute in the form of i) a sugar that has solubility of at least 100 g/g water, preferably at least 300 g/g water, at 20° c and 1 atm pressure; or ii) a sugar that is not glucose.

13. A composition according to claim 12, wherein the solute is, or comprises, fructose.

14. A composition according to any of claims 11 to 13, wherein there is 10% or less, by weight, of the solute in the composition.

15. A composition according to any of claims 11 to 14, wherein there is 5% or less, by weight, of the solute in the composition.

16. A composition according to any of claims 11 to 15, wherein there is at least 1%, by weight, of the solute in the composition.

17. A composition according to any preceding claim, comprising non-aqueous solvent.

18. A composition according to claim 17, wherein the non-aqueous solvent is, or comprises, a polyol, preferably wherein the polyol is glycerol.

19. A composition according to any of claims 17 to 18, wherein there is at least 10%, at least 50%, at least 60%, at least 70%, or at least 80% by weight of the non-aqueous solvent in the composition; and/or there is 60% or less, 50% or less, 40% or less, or 30% or less, by weight of the non-aqueous solvent in the composition.

20. A composition according to any of claims 17 to 18, wherein there is 20% to 75%, by weight, preferably 25% to 70% by weight of the non-aqueous solvent in the composition.

21. A composition according to any preceding claim, comprising antioxidant.

22. A composition according to claim 21, wherein the antioxidant is ascorbic acid, tocopherol or ascorbyl palmitate, preferably ascorbic acid.

23. A composition according to any of claims 21 to 22, wherein there is at least 0.05%, preferably at least 0.1%, by weight of the antioxidant in the composition.

24. A composition according to any of claims 21 to 23, wherein there is 2% or less by weight, preferably 1% or less by weight of the antioxidant in the composition.

25. A composition according to any preceding claim, which comprises substantially no hydrogen peroxide.

26. A composition according to any preceding claim, wherein there is less than 10 ppm hydrogen peroxide in the composition.

27. A composition according to claim any preceding claim, wherein there is less than 6 ppm hydrogen peroxide in the composition.

28. A composition according to any preceding claim, wherein there is less than 3 ppm hydrogen peroxide in the composition.

29. A composition according to any preceding claim, wherein the polymer is or comprises polyethylene glycol.

30. A composition according to any preceding claim, wherein there is at least 30%, or at least 60%, by weight, of the polymer in the composition.

31. A composition according to any preceding claim, wherein there is 70% or less by weight of the polymer in the composition.

32. A composition according to any preceding claim, wherein the polymer comprises a first polymer and a second polymer, and the first polymer has a lower molecular weight than the second polymer.

33. A composition according to claim 32, wherein the polymer comprises a third polymer of intermediate molecular weight to the first and second polymer.

34. A composition according to claim 33, wherein the composition comprising a first PEG polymer of molecular weight less than 1000, a second PEG polymer of molecular weight of 1000-3000, and a third PEG polymer of molecular weight greater than 3000.

35. A composition according to any preceding claim, comprising a buffer, optionally a TRIS buffer.

36. A composition according to any preceding claim, which is at a pH of 6.5 to 7.5.

37. A composition according to any preceding claim which is sterile.

38. A composition according to any preceding claim, comprising substantially no zinc oxide.

39. A composition according to any preceding claim, comprising substantially no catalase.

40. A composition according to any preceding claim, comprising substantially no peroxidase.

41. A composition according to any preceding claim, comprising substantially no lactoferrin

42. A composition according to any preceding claim, wherein the enzyme is a purified enzyme.

43. A composition according to claim 42, wherein the enzyme has a mass purity at least 95%, preferably at least 98%.

44. A composition according to any preceding claim, wherein the substrate is a purified substrate.

45. A composition according to claim 44, wherein the substrate has a mass purity of at least 95%, preferably at least 98%.

46. A composition according to claim 11 or any claim dependent on claim 11, wherein the solute is a purified solute

47 A composition according to claim 46, wherein the solute has a mass purity of at least 95%, preferably at least 98%.

48. A composition according to any preceding claim, wherein there is 0.001% to 0.5%, by weight, of the enzyme in the composition, or 0.0025% to 0.2%, by weight, of the enzyme in the composition.

49. A composition according to any preceding claim, which does not comprise honey.

50. A composition according to any preceding claim with a viscosity of 12000 to 15000 MPas at 20° C. and 1 atm.

51. A composition according to any preceding claim which provides for release of hydrogen peroxide at a concentration of 750 to 2,500 μM, at one hour, 24 hours, and/or 72 hours following a 1:1 (by weight) dilution of the composition with water.

52. A composition according to any preceding claim, which at 24 hours, following a 1:1 dilution (by weight) with water, the level of hydrogen peroxide production increases by a factor of at least 10.

53. A wound dressing which comprises a dressing material for dressing a wound, and a composition according to any preceding claim, optionally wherein the wound dressing material comprises carboxymethylcellulose.

54. A package or container comprising a composition as defined in any of claims 1 to 52.

55. A package or container according to claim 54, which is air-tight.

56. A dispensing device containing a composition as defined in any of claims 1 to 52.

57. A dispensing device according to claim 56, wherein the dispensing device is a spray, a tube or a syringe.

58. A composition according to any of claims 1 to 52, for use as a medicament.

59. A composition according to any of claims 1 to 52, for use in prevention, treatment or amelioration of a microbial infection.

60. A composition according to any of claims 1 to 52, for use in treatment of a wound.

61. A method of preparing a liquid or gel composition, optionally wherein the composition is as defined in claim 1 or any claim dependent on claim 1, the method comprising mixing: enzyme that is able to convert a substrate to release hydrogen peroxide; and substrate for the enzyme,
wherein the composition is formulated such that there is not sufficient free water to allow the enzyme to convert the substrate, or is formulated to have a water activity of 0.7 or less, and wherein:
A) the method comprises adding polymer, and i) the polymer is added such that it is greater than 50% by weight of the composition, and/or ii) the composition is formulated such that there is less than 20% by weight of sugar in the composition; or
B) the method comprises adding non-aqueous solvent and not adding honey, wherein the substrate is added such that it is 5% or less by weight of the composition, and wherein i) the non-aqueous solvent is added such that it is greater than 50% by weight of the composition, and/or ii) the composition is formulated such that there is less than 20% by weight of sugar in the composition.

62. A method according to claim 61, wherein the polymer is added such that it is greater than 50% by weight of the composition.

63. A method according to any of claims 61 to 62, wherein the composition is formulated such that there is less than 10% by weight of water in the composition.

64. A method according to any of claims 61 to 63, wherein the composition is formulated such that there is 10% or less, by weight, of sugar in the composition.

65. A method according to any of claims 61 to 64, wherein the composition is formulated such that there is 6% or less, by weight, of sugar in the composition.

66. A method according to any of claims 61 to 65, wherein the composition is formulated such that the water activity (a w) of the composition is 0.6 or less, preferably 0.5 or less.

67. A method according to any of claims 61 to 66, wherein the composition is formulated such that the substrate is 10% or less, by weight, of the composition.

68. A method according to any of claims 61 to 67, wherein the composition is formulated such that the substrate is 5% or less, by weight, of the composition.

69. A method according to any of claims 61 to 68, wherein the composition is formulated such that the substrate is at least 1%, by weight, of the composition.

70. A method according to any of claims 61 to 69, wherein the substrate is, or comprises, glucose oxidase.

71. A method according to any of claims 61 to 70, comprising mixing a solute in the form of a sugar that has solubility of 100 g/g water at 20° C. and 1 atm pressure, or a solute in the form of a sugar that is not glucose.

72. A method according to claim 71, wherein the solute is, or comprises, fructose.

73. A method according to claim 71 or claim 72, wherein the solute is added such that it is less than 20%, by weight, of the composition.

74. A method according to any of claims 71 to 73, wherein the solute is added such that it is 10% or less, by weight, of the composition.

75. A method according to any of claims 71 to 74, wherein the solute is added such that it is 5% or less, by weight, of the composition.

76. A method according to any of claims 71 to 75, wherein the solute is added such that it is at least 1%, by weight, of the composition.

77. A method according to any of claims 61 to 76, comprising adding a non-aqueous solvent.

78. A method according to claim 77, wherein the non-aqueous solvent is, or comprises, a polyol, preferably wherein the polyol is glycerol.

79. A method according to any of claims 77 to 78, wherein the non-aqueous solvent is added such that it is at least at least 10%, at least 50%, at least 60%, at least 70% or at least 80%, by weight, of the composition.

80. A method according to any of claims 77 to 79, wherein the non-aqueous solvent is added such that it is up to 30% by weight of the composition.

81. A method according to any of claims 61 to 80, comprising mixing antioxidant.

82. A method according to claim 81, wherein the antioxidant is ascorbic acid, tocopherol or ascorbyl palmitate, preferably ascorbic acid.

83. A method according to any of claims 81 to 82, wherein the antioxidant is added such that it is at least 0.05%, preferably at least 0.1%, by weight of the composition.

84. A method according to any of claims 81 to 83, wherein the antioxidant is added such that it is 2% or less by weight, preferably 1% or less by weight, of the composition

85. A method according to any of claims 61 to 84, wherein the composition comprises substantially no hydrogen peroxide.

86. A method according to any of claims 61 to 85, wherein hydrogen peroxide is present at a concentration of less than 10 ppm in the composition.

87. A method according to any of claims 61 to 86, wherein hydrogen peroxide is present at a concentration of 6 ppm or less in the composition.

88. A method according to any of claims 61 to 87, wherein hydrogen peroxide is present at a concentration of 3 ppm or less in the composition.

89. A method according to any of claims 61 to 88, wherein the polymer is or comprises polyethylene glycol.

90. A method according to any of claims 61 to 89, wherein the polymer is added such that it is at least 60% by weight of the composition.

91. A method according to any of claims 61 to 90, wherein the polymer is added such that it is 70% or less by weight of the composition.

92. A method according to any of claims 61 to 91, wherein the polymer comprises a first polymer and a second polymer, and the first polymer has a lower molecular weight than the second polymer.

93. A method according to claim 92, wherein the polymer comprises a third polymer of intermediate molecular weight to the first and second polymer.

94. A method according to claim 93, wherein the composition comprising a first PEG polymer of molecular weight less than 1000, a second PEG polymer of molecular weight of 1000-3000, and a third PEG polymer of molecular weight greater than 3000.

95. A method according to any of claims 61 to 94, comprising mixing a buffer, optionally a TRIS buffer.

96. A method according to any of claims 61 to 95, comprising formulating the composition such that it is at a pH of 6.5 to 7.5.

97. A method according to any of claims 61 to 96, comprising a sterilisation step, to form a sterile composition, optionally wherein the sterilisation step comprises expose to gamma or electron beam radiation.

98. A method according to any of claims 61 to 97, wherein the enzyme is a purified enzyme.

99. A method according to claim 98, wherein the enzyme has a mass purity at least 95%, preferably at least 98%.

100. A method according to any of claims 61 to 99, wherein the substrate is a purified substrate.

101. A method according to claim 100, wherein the substrate has a mass purity of at least 95%, preferably at least 98%.

102. A method according to claim 71 or any claim dependent on claim 71, wherein the solute is a purified solute

103. A method according to claim 102, wherein the solute has a mass purity of at least 95%, preferably at least 98%.

104. A method according to any of claims 61 to 103, wherein the enzyme is added such that it is 0.001% to 0.5%, by weight, of the composition.

105. A method according to any of claims 61 to 104, wherein the enzyme is added such that it is 0.0025% to 0.2%, by weight, of the composition.

106. A method according to any of claims 61 to 105, wherein the enzyme is added such that it is 25 to 2000 ppm of the composition.

107. A method according to any of claims 61 to 106, which does not comprise adding honey.

108. A method according to any of claims 61 to 107, in which the composition is formulated to have a viscosity of 12000 to 15000 MPas at 20 ° C. and 1 atm.

109. A method according to any of claims 61 to 108, which comprises adding substantially no: zinc oxide, catalase, peroxidase, and/or lactoferrin.

110. Use of a composition according to any of claims 1 to 52 in the manufacture of a medicament for the prevention, treatment, or amelioration of an infection.

111. A method of preventing, treating, or ameliorating an infection, which comprises administering an effective amount of a composition according to any of claims 1 to 52.

112. Use of a composition according to any of claims 1 to 52 in the manufacture of a medicament for the treatment of a wound.

113. A method of treating a wound, which comprises administering an effective amount of a composition according to any of claims 1 to 52.
